(19) United States
(12) Patent Application Publication
(10) Pub. No.: US 2019/0062827 A1
(43) Pub. Date: Feb. 28, 2019

(54) HYPER-THERMOSTABLE LYSINE-MUTANT ssDNA/RNA LIGASES

(71) Applicant: RGENE, Inc., San Francisco, CA (US)

(72) Inventors: Yu Zheng, San Francisco, CA (US); Manqing Hong, San Francisco, CA (US)

(21) Appl. No.: 16/082,500

(22) PCT Filed: Mar. 14, 2017
(86) PCT No.: PCT/US17/22232

§ 371 Date: Sep. 5, 2018

Related U.S. Application Data
(60) Provisional application No. 62/307,658, filed on Mar. 14, 2016.

(51) Int. Cl. C12Q 1/6869; C12N 9/00; C12Q 1/686; C07H 21/04

(52) U.S. Cl. CPC C12Q 1/6869; C12N 9/93; C12Y 600/00; C07H 21/04; C12Q 1/686

(57) ABSTRACT

Provided herein are compositions, systems, and methods employing hyper-thermostable lysine-mutant ssDNA/RNA ligases that possesses both ssRNA ligase and ssDNA ligase activity. In certain embodiments, such hyper-thermostable lysine-mutant ssDNA/RNA ligases are used to ligate an first single stranded nucleic acid sequence with a 5′ adenylated end to a second single stranded nucleic acid sequence (e.g., at a temperature of at least 75° C.) to form a ligated nucleic acid sequence. In further embodiments, the ligated nucleic acid sequence is sequenced.

The present application claims priority to U.S. Provisional application Ser. No. 62/307,658, filed Mar. 14, 2016, which is herein incorporated by reference in its entirety.

### FIELD

Provided herein are compositions, systems, and methods employing hyper-thermostable lysine-mutant ssDNA/RNA ligases that possesses both ssRNA ligase and ssDNA ligase activity. In certain embodiments, such hyper-thermostable lysine-mutant ssDNA/RNA ligases are used to ligate a first single-strand nucleic acid sequence with a 5′ adenylated end to a second single-strand nucleic acid sequence (e.g., at a temperature of at least 75° C.) to form a ligated nucleic acid sequence. In further embodiments, the ligated nucleic acid sequence is amplified and/or sequenced.

### BACKGROUND

DNA or RNA ligases are divalent metal ion dependent enzymes that utilize ATP or NAD+ to catalyze phosphodiester bond formation between adjacent polynucleotide termini possessing a 3′-hydroxyl and a 5′-phosphate (Tomkinson et al., PNAS, 2006; Silber et al, PNAS, 1972). Depending on their origin of species, natural occurring DNA or RNA ligases may have many unique properties, for example, substrate specificity, sequence and domain organization, optimal reaction condition such as pH, co-factor dependence, temperature and salt tolerance etc. Their activities in vitro have also been exploited in numerous molecular biology protocols, making them critical tools for modern biotechnology.

All known DNA and RNA ligases perform the catalysis via a common pathway which involves three nucleotidyl transfer reactions (Lehman et al, Science, 1974; Lindahl et al, Annu Rev Biochem, 1992). In the case of ATP-dependent DNA or RNA ligases, the first step (step 1) involves the attack on the a-phosphate of ATP by ligase, which results in release of pyrophosphate and formation of a ligase-AMP intermediate. AMP is linked covalently to the amino group of a lysine residue within a conserved sequence motif. In the second step (step 2), the AMP nucleotide is transferred to the 5′-phosphate-terminated DNA or RNA strand to form a 5′-App-DNA/RNA intermediate. In the third and final step (step 3), attack by the 3′-OH strand on the 5′-App-DNA/RNA end joins the two polynucleotides and liberates AMP.

DNA ligases catalyze the formation of phosphodiester bonds at single-stranded nicks in double-stranded DNA. In vivo, this activity is critical for maintaining genomic integrity during DNA replication, DNA recombination, and DNA excision repair. Among them, T4 DNA ligase has been widely used in vitro to join double-stranded breaks with cohesive and blunt ends. RNA ligases are typically classified into at least two broad families. The Rnl1 family includes the T4 RNA ligase 1 (Rnl1) and the tRNA ligases from fungi, yeasts, and plants. These enzymes repair breaks in single-stranded RNA. The Rnl2 family is represented by the bacteriophage T4 RNA ligase 2 (T4Rnl2) and the RNA-editing ligases from the protozoans Trypanosoma and Leishmania (Ho and Shuman, PNAS, 2002). In vivo, these enzymes primarily seal nicks in double-stranded RNA. In vitro, T4Rnl2 and its mutants have been shown to join single-stranded RNA (Yin S et al, JBC, 2003), as well as between 3′-end of single-strand RNA and 5′-end of single-strand DNA adaptor (Viollet et al, 2011, BMC Biotechnol).

It has been observed that single-strand RNA ligases can sometimes accept single-strand DNA as substrate, albeit sometimes with lower ligation efficiency. For example, T4 RNA ligase 1 (T4Rnl1) has been known for a long time and used to ligate single-strand RNA (ssRNA), and ssDNA with a lower efficiency (Lau et al., 2001). Homologs within the T4 RNA ligase 1 family exhibit single-strand DNA ligation activity. For example, thermostable single-strand DNA ligase from Thermus scotoductus bacteriophage TS2126, which is a T4Rnl1 homolog, can ligate ssDNA at elevated temperature around 65° C. (Blondal et al, NAR, 2005) and was marketed as CircLigase by EpiCenter Biotechnologies (WO2004/027056A2). Another T4Rnl1 homolog from Rhodothermus marinus bacteriophage RM378 was also reported to ligate both ssRNA and ssDNA (Blondal et al, NAR, 2002). Due to its unique ssDNA ligation activity at relatively high temperature (˜65° C.), CircLigase quickly finds its application in many applications, such as in the process of library preparation of high-throughput next-generation sequencing (Gansauge M T, Nat. Protol., 2013).

Advancement in next-generation sequencing (NGS) has transformed biomedical and clinical research with an accelerating momentum. One of the most crucial families of enzymes used in the NGS library preparation workflow is ligases. The significance of the ligation step is that it attaches short pieces of DNA or RNA adaptors with known sequences to the ends of the unknown library sequences, thus provide priming sites to facilitate downstream steps such as PCR or primer extension. For DNA-sequencing applications, ligation step in the NGS library preparation workflow commonly occur between double-stranded library nucleic acids fragments and double-strand adaptor, but also possible between single-stranded nucleic acids fragments and 5′-phosphorylated single-stranded adaptor. The double-stranded ligation reaction is oftentimes catalyzed by T4 DNA ligase and is usually carried out at ambient or even lower (e.g., 16° C.) temperature. Single-stranded DNA ligation is currently catalyzed by enzymes such as CircLigase, with reaction temperature as high as 65° C. (Gansauge M T, Nat. Protol., 2013). In other applications such as small RNA sequencing, T4Rnl1 and T4Rnl2 mutants are commonly used to attached single-stranded DNA adaptor to single-strand RNA library fragments, usually at 37° C. or lower.

### SUMMARY

Provided herein are compositions, systems, and methods employing hyper-thermostable lysine-mutant ssDNA/RNA ligases that possesses both ssRNA ligase and ssDNA ligase activity. In certain embodiments, such hyper-thermostable lysine-mutant ssDNA/RNA ligases are used to ligate an first single stranded nucleic acid sequence with a 5′ adenylated end to a second single stranded nucleic acid sequence (e.g., at a temperature of at least 75° C.) to form a ligated nucleic acid sequence. In further embodiments, the ligated nucleic acid sequence is amplified and/or sequenced.

In some embodiments, provided herein are methods of ligating single-stranded nucleic acid comprising: a) combining in a reaction mixture: i) a hyper-thermostable lysine-mutant ssDNA/RNA ligase which is a mutated version of a precursor hyper-thermostable ssRNA ligase (e.g., a precursor ssRNA ligase shown in Table 2, or truncated or mutated versions thereof), wherein the precursor hyper-thermostable ssRNA ligase has a Motif I EKx(D/N/H)G (SEQ ID NO:32) and possess ssRNA ligase activity, but not ssDNA ligase activity, at a temperature of at least 75° C. (e.g., at least 75 . . . 85 . . . 95 . . . or 100° C., or between 75-100° C.), and wherein the hyper-thermostable lysine-mutant ssDNA/RNA ligase has an amino acid substitution at the K (lysine) in the Motif I, and possesses both ssRNA ligase and ssDNA ligase activity at a temperature of at least 75° C. (e.g., at least 75 . . . 85 . . . 95 . . . or or 100° C., or between 75-100° C.), ii) a first single stranded nucleic acid sequence with a 5′ end base that is adenylated (or becomes adenylated while in the reaction mixture), and iii) a second single stranded nucleic acid sequence with a 3′ end base; and b) incubating the reaction mixture at a temperature of at least 75° C. (e.g., at least 75 . . . 85 . . . 95 . . . or 100° C., or between 75-100° C.) such that the hyper-thermostable lysine-mutant ssDNA/RNA ligase ligates the 5′ adenylated end of the first single stranded nucleic acid sequence to the 3′ end of the second single stranded nucleic acid sequence to form a ligated nucleic acid sequence. In certain embodiments, the incubating the reaction mixture is at a temperature of at least 85° C. or at least 95° C. In particular embodiments, the hyper-thermostable lysine-mutant ssDNA/RNA ligase is an adenylation-deficient ATP-dependent ligase (i.e., a ligase that cannot form the AMP-ligase intermediate by reacting with ATP).

In particular embodiments, provided herein are systems and kits comprising: a) a hyper-thermostable lysine-mutant ssDNA/RNA ligase which is a mutated version of a precursor hyper-thermostable ssRNA ligase (e.g., a precursor ssRNA ligase shown in Table 2, or truncated or mutated versions thereof), wherein the precursor hyper-thermostable ssRNA ligase has a Motif I EKx(D/N/H)G (SEQ ID NO:32) and possess ssRNA ligase activity, but not ssDNA ligase activity, at a temperature of at least 75° C., and wherein the hyper-thermostable lysine-mutant ssDNA/RNA ligase has an amino acid substitution at the K (lysine) in the Motif I, and possesses both ssRNA ligase and ssDNA ligase activity at a temperature of at least 75° C., b) a first single stranded nucleic acid sequence with a 5′ end base that is adenylated; and c) a second single stranded nucleic acid sequence with a 3′ end base. In particular embodiments, the kits and systems further comprise a first container and a second container, and wherein the hyper-thermostable lysine-mutant ssDNA/RNA ligase is in the first container, and the first and/or second single-stranded nucleic acid sequence is in the second container.

In certain embodiments, provided herein are compositions comprising: a hyper-thermostable lysine-mutant ssDNA/RNA ligase which is a mutated version of a precursor hyper-thermostable ssRNA ligase (e.g., a precursor ssRNA ligase shown in Table 2 or truncated or mutated versions thereof), wherein the precursor hyper-thermostable ssRNA ligase has a Motif I EKx(D/N/H)G (SEQ ID NO:32) and possess ssRNA ligase activity, but not ssDNA ligase activity, at a temperature of at least 75° C. (e.g., at a temperature between 75-85 or 75-100° C.), and wherein the hyper-thermostable lysine-mutant ssDNA/RNA ligase has an amino acid substitution at the K (lysine) in the Motif I, and possesses both ssRNA ligase and ssDNA ligase activity at a temperature of at least 75° C. (e.g., at a temperature between 75-85 or 75-100° C.). In particular embodiments, the compositions further comprise at least one of the following: a) a first single stranded nucleic acid sequence with a 5′ end base that is adenylated, and b) a second single stranded nucleic acid sequence with a 3′ end base.

In particular embodiments, the 5′ end base of the first single stranded nucleic acid sequence is a DNA base, and wherein the 3′ end base of the second single stranded nucleic acid sequence is an RNA base or a DNA base. In other embodiments, the 5′ end base of the first single stranded nucleic acid sequence is a DNA base or an RNA base, and wherein the 3′ end base of the second single stranded nucleic acid sequence is a DNA base. In some embodiments, all or nearly all of the bases in the first single stranded nucleic acid sequence are DNA bases. In particular embodiments, all of the bases, or nearly all of the bases, in the second single stranded nucleic acid sequence are DNA bases.

In certain embodiments, the amino acid substitution for the K (lysine) is an amino acid selected from the group consisting of: alanine (A), serine (S), cysteine (C), valine (V), threonine (T), and Glycine (G). In other embodiments, the precursor hyper-thermostable ssRNA ligase is a wild-type hyper-thermostable ssRNA ligase. In further embodiments, the wild-type hyper-thermostable ssRNA ligase is from a species selected from the group consisting of: Thermococcus kodakarensis, Pyrococcus, yayanosii, Pyrococcus horikoshii, Pyrococcus abyssi, Pyrococcus furiosus, Hyperthermus butylicus, Aeropyrum pernix, Staphylothermus marinus, Pyrolobus fumarii, and Aquifex aeolicus. In additional embodiments, the hyper-thermostable lysine-mutant ssDNA/RNA ligase is encoded by one of the amino acid sequences shown in SEQ ID NO:1-11. In further embodiments, the hyper-thermostable lysine-mutant ssDNA/RNA ligase is encoded by an amino acid sequence that has at least 85%, 95%, or 99% sequence identity to one of the amino acid sequences shown in SEQ ID NO:1-11 (e.g., at least 85% . . . 90% . . . 95% . . . 98% . . . 99% . . . 99.5%). In certain embodiments, the hyper-thermostable lysine-mutant ssDNA/RNA ligase is encoded by an N-terminal or C-terminal truncated amino acid sequence shown in SEQ ID NO:1-11 (e.g., where the SEQ ID NO is missing 1-40 amino acids at either the N or C terminal, yet still has the same ligase activity as the full length sequence).

In certain embodiments, the hyper-thermostable lysine-mutant ssDNA/RNA ligase comprises a Motif sequence selected from the group consisting of: EGx(D/N/H)G (SEQ ID NO:12), EPx(D/N/H)G (SEQ ID NO:13), EAx(D/N/H)G (SEQ ID NO:14), EVx(D/N/H)G (SEQ ID NO:15), ELx(D/N/H)G (SEQ ID NO:16), E Ix(D/N/H)G (SEQ ID NO:17), EMx(D/N/H)G (SEQ ID NO:18), ECx(D/N/H)G (SEQ ID NO:19), EFx(D/N/H)G (SEQ ID NO:20), EYx(D/N/H)G (SEQ ID NO:21), EWx(D/N/H)G (SEQ ID NO:22), EHx(D/N/H)G (SEQ ID NO:23), ERx(D/N/H)G (SEQ ID NO:24), EQx(D/N/H)G (SEQ ID NO:25), ENx(D/N/H)G (SEQ ID NO:26), EEx(D/N/H)G (SEQ ID NO:27), EDx(D/N/H)G (SEQ ID NO:28), ESx(D/N/H)G (SEQ ID NO:29), ETx(D/N/H)G (SEQ ID NO:30) and Ex(D/N/H)G (SEQ ID NO:31), point deletion of lysine); wherein x is any amino acid. In particular embodiments, x is an amino acid with a small or smaller side chain compared to other amino acids.

In particular embodiments, the first single stranded nucleic acid sequence is a sequencing adapter (e.g., a next generation sequence adapter). In some embodiments, the second single stranded nucleic acid sequence comprises a sequencing library fragment. In particular embodiments, the methods further comprising: c) sequencing the ligated nucleic acid sequence.

In some embodiments, provided herein are methods comprising: a) combining in a reaction mixture: i) a single-stranded RNA ligase from Pyrococcus furiousus (PfuRnl2, WP_014835129.1) (or truncated or mutated version thereof), ii) a single-stranded nucleic acid sequence with an un-adenylated 5′ end, and iii) ATP molecules; and b) incubating the reaction mixture under condition such that the PfuRnl2 adenylates the un-adenylated 5′ end of the single-stranded nucleic acid sequence, thereby generating a first single stranded nucleic acid sequence with a 5′ end base that is adenylated. In particular embodiments, the incubating is at a temperature of at least 75° C. (e.g., at least 75 . . . 85 . . . 95 . . . or 100° C.; or between 75-85 or between 75-100° C.). In further embodiments, the combining in the reaction mixture further includes: iv) a second single stranded nucleic acid sequence with a 3′ end base, v) a hyper-thermostable lysine-mutant ssDNA/RNA ligase which is a mutated version of a precursor hyper-thermostable ssRNA ligase, wherein the precursor hyper-thermostable ssRNA ligase has a Motif I EKx(D/N/H)G (SEQ ID NO:32) and possess ssRNA ligase activity, but not ssDNA ligase activity, at a temperature of at least 75° C., and wherein the hyper-thermostable lysine-mutant ssDNA/RNA ligase has an amino acid substitution at the K (lysine) in the Motif I, and possesses both ssRNA ligase and ssDNA ligase activity at a temperature of at least 75° C. In some embodiments, the hyper-thermostable lysine-mutant ssDNA/RNA ligase ligates the 5′ adenylated end of the first single stranded nucleic acid sequence to the 3′ end of the second single stranded nucleic acid sequence to form a ligated nucleic acid sequence.

In certain embodiments, provided herein are compositions, kits, or systems comprising: a) a single-stranded RNA ligase from Pyrococcus furiousus (PfuRnl2, WP_014835129.1) (or truncated or mutated version thereof with the same or similar activity), and b) a hyper-thermostable lysine-mutant ssDNA/RNA ligase which is a mutated version of a precursor hyper-thermostable ssRNA ligase, wherein the precursor hyper-thermostable ssRNA ligase has a Motif I EKx(D/N/H)G (SEQ ID NO:32) and possess ssRNA ligase activity, but not ssDNA ligase activity, at a temperature of at least 75° C., and wherein the hyper-thermostable lysine-mutant ssDNA/RNA ligase has an amino acid substitution at the K (lysine) in the Motif I, and possesses both ssRNA ligase and ssDNA ligase activity at a temperature of at least 75° C. In other embodiments, the compositions, kits, and systems further comprise: c) a single-stranded nucleic acid sequence with an un-adenylated 5′ end.

### BRIEF DESCRIPTION OF THE DRAWINGS

FIG. 1 shows activities of wild-type PhoRnl2 (A and B), PfuRnl2 (C and D), HbuRnl2 (E and F) on 5′-phosphorylated single-stranded (ss) RNA and DNA in varying concentration of ATP. (A) Activity of PhoRnl2 on 5′-phosphorylated ssRNA. Lane 1, 100 uM ATP; lane 2, 10 uM ATP; lane 3, 1 uM ATP; lane 4, no ATP added. pRNA, 5′-phosphorylated ssRNA; AppRNA, 5′-adenylated ssRNA; circRNA, circular ssRNA. (B) Activity of PhoRnl2 on 5′-phosphorylated ssDNA. Lane 1, 100 uM ATP; lane 2, 10 uM ATP; lane 3, 1 uM ATP; lane 4, no ATP added. pDNA, 5′-phosphorylated ssDNA; AppDNA, 5′-adenylated ssDNA. (C) Activity of PfuRnl2 on 5′-phosphorylated ssRNA. Lane 1, 100 uM ATP; lane 2, 10 uM ATP; lane 3, 1 uM ATP. (D) Activity of PfuRnl2 on 5′-phosphorylated ssDNA. Lane 1, 100 uM ATP; lane 2, 10 uM ATP; lane 3, 1 uM ATP. (E) Activity of HbuRnl2 on 5′-phosphorylated ssRNA. Lane 1, 100 uM ATP; lane 2, 10 uM ATP; lane 3, 1 uM ATP. (F) Activity of HbuRnl2 on 5′-phosphorylated ssDNA. Lane 1, 100 uM ATP; lane 2, 10 uM ATP; lane 3, 1 uM ATP. All reactions were incubated at 90° C. for 1 hour and resolved on 20% polyacrylamide denaturing gel containing 7M urea. Gels were stained using Sybr Gold and visualized using GE ImageQuant LAS scanner.

FIG. 2 shows activity of wild-type HbuRnl2 on 5′-adenylated ssDNA with and without ATP. Lane 1, 5′-adenylated ssDNA with purified wild-type HbuRnl2 and 100 uM ATP; lane 2, 5′-adenylated ssDNA with purified wild-type HbuRnl2 and no ATP; lane 3, 5′-adenylated ssDNA only. All reactions were incubated at 90° C. for 1 hour and resolved on 20% polyacrylamide denaturing gel containing 7M urea. Gels were stained using Sybr Gold and visualized using GE ImageQuant LAS scanner.

FIG. 3 shows the activity of mutant HbuRnl2K106A on 5′-adenylated ssDNA. Phosphorylated ssDNA was first adenylated using 5′-adenylation kit (NEB#E2610) and the product was purified from reaction (shown in Lane 5). In a 10 ul total reaction volume, reaction buffer contains 70 mM Tris-HCl (pH=7.6), 10 mM MgCl₂, 5 mM DTT. Lane 1, 1 ul 5′-adenylated ssDNA with 1 ul HbuRnl2K106A, incubated at 90° C. for 1 hour. Lane 2, 1 ul 5′-adenylated ssDNA with 1 ul HbuRnl2K106A, incubated at 90° C. for 1 hour, after which 1 ul Exonuclease I (NEB#M0293) was added and further incubated at 37° C. for 1 hour. Lane 3, 1 ul 5′-adenylated ssDNA with 1 ul MthRnl (NEB#E2610), incubated at 65° C. for 1 hour. Lane 4, 1 ul 5′-adenylated ssDNA with 1 ul MthRnl (NEB#E2610), incubated at 65° C. for 1 hour, after which 1 ul Exonuclease I (NEB#M0293) was added and further incubated at 37° C. for 1 hour. All reactions were run on 20% polyacrylamide denaturing gel containing 7M urea.

FIG. 4 shows the activity of the HbuRnl2 lysine 106 mutants. All mutants were His-tagged and purified using Ni-NTA beads. Purified mutants were assayed for their ssDNA ligation activity using 5′-adenylated ssDNA. 1 ul purified HbuRnl2K106A mutant was added to a 10 ul ligation containing 70 mM Tris-HCl, 5 mM DTT, 5 mM MnCl₂ (pH 7.6@25° C.) and 10 pmol of adenylated ssDNA substrate. The reactions were incubated at 90° C. for 1 hour and resolved on 20% denaturing polyacrylamide gel with 7M urea. The gel was stained using SYBR Gold and scanned using GE ImageQuant LAS scanner. Circularized ssDNA band was labeled at the right of the gel. Disappearance of the adenylated band and the accumulation of the circularized band suggest the activity of the mutant. Lanes from 1 to 18 correspond to mutants: 1=K106L; 2=K106I; 3=K106W; 4=K106M; 5=K106T; 6=K106X (point deletion); 7=K106H; 8=K106G; 9=K106N; 10=K106Y; 11=K106V; 12=K106P; 13=K106C; 14=K106S; 15=K106E; 16=K106F; 17=K106R; 18=K106A.

FIG. 5 shows the reaction temperature profile of the mutant HbuRnl2K106A. Ligation using 5′-adenylated ssDNA were conducted in Bio-rad MyCycler using temperature gradient option. All reactions were run on 20% polyacrylamide denaturing gel containing 7M urea. The gel was stained using Sybr Gold and scanned using GE Imager LAS. The gel image was analyzed by ImageJ. The circularized ssDNA band was converted to numerical values, normalized by the value at 70° C., and plotted against reaction temperature (X-axis). The data points were fitted by using a linear curve fitting.

FIG. 6 shows the inter-molecule ligation between two pieces of ssDNA using HbuRnl2K106A. Ligation reactions were done in 10 ul volume, with buffer containing 70 mM Tris-HCl (pH=7.6), 10 mM MgCl₂, 5 mM DTT, 5 mM Mn²⁺. Each reaction contains 2 pmol short ssDNA (lowest band) and about 20 pmol 5′-adenylated 3′-NH2 blocked adaptor (top of the middle two bands, the lower band is phosphorylated adaptor, partly due to incomplete adenylation). In addition, lane 1 contains 12% PEG8000 and lane 2 contains 15% PEG8000. Both reactions were incubated at 90° C. for 1 hour and resolved on 20% polyacrylamide denaturing gel containing 7M urea. Gels were stained using Sybr Gold and visualized using GE ImageQuant LAS scanner.

FIG. 7 shows exemplary ssDNA ligation based NGS library preparation.

FIG. 8 shows the use of PfuRnl2 and HbuRnl2K106A as a mixture. All 10 ul reactions were done in 70mM Tris-HCl (pH=7.6), 10 mM MgCl₂, 5 mM DTT. Reactions were incubated at 90C for 1 hour and resolved on 20% polyacrylamide denaturing gel containing 7M urea. Gels were stained using Sybr Gold and visualized using GE ImageQuant LAS scanner. Lane 3, 5′-phosphorylated DNA only; lane 1, 5′-phosphorylated DNA with 1 ul PfuRnl2 and 1 ul HbuRnl2K106A and 100 uM ATP; lane 2, 5′-phosphorylated DNA with 1 ul PfuRnl2 and 1 ul HbuRnl2K106A and 10 uM ATP.

### DEFINITIONS

In this application, the 5′-adenylated end of ssDNA or ssRNA if referred to as “donor” the end, and the 3′-OH end of ssDNA or ssRNA is referred to as the “acceptor” end.

### DETAILED DESCRIPTION

Provided herein are compositions, systems, and methods employing hyper-thermostable lysine-mutant ssDNA/RNA ligases that possess both ssRNA ligase and ssDNA ligase activity. In certain embodiments, such hyper-thermostable lysine-mutant ssDNA/RNA ligases are used to ligate a first single stranded nucleic acid sequence with a 5′ adenylated end to a second single stranded nucleic acid sequence (e.g., at a temperature of at least 75° C.) to form a ligated nucleic acid sequence. In further embodiments, the ligated nucleic acid sequence is amplified and/or sequenced.

In certain embodiments, the hyper-thermostable lysine-mutants are as provided in SEQ ID Nos:1-11 in Table 1 below, or N or C terminal truncated, versions thereof.

TABLE 1
Hyper-thermostable Lysine-Mutant ssDNA/RNA Ligases

| | SEQ ID | |
|---|---|---|
| Amino Acid Sequence | NO: | Species name |
| MVSSYFRNLLLKLGLPEERLEVLEGKGALAEDEFEGIRYVRFRDSARNFRRG | 1 | Thermococcus |
| TVVFETGEAVLGFPHIKRVVQLENGIRRVFKNKPFYVEEXVDGYNVRVVKVK | | kodakarensis |
| DKILAITRGGFVCPFTTERIEDFVNFDFFKDYPNLVLVGEMAGPESPYLVEG | | |
| PPYVKEDIEFFLFDIQEKGTGRSLPAEERYRLAEEYGIPQVERFGLYDSSKV | | |
| GELKELIEWLSEEKREGIVMKSPDMRRIAKYVTPYANINDIKIGSHIFFDLP | | |
| HGYFMGRIKRLAFYLAENHVRGEEFENYAKALGTALLRPFVESIHEVANGGE | | |
| VDETFTVRVKNITTAHKMVTHFERLGVKIHIEDIEDLGNGYWRITFKRVYPD | | |
| ATREIRELWNGLAFVD | | |
| Where X is any amino acid except K. | | |
| MVSSHFKEILMRLGLPEDRIEVLEAKGGITEEEFDGIRYLRFKDSARGLRRG | 2 | Pyrococcus yayanosii |
| TVVFDEANVILGFPHIKRVVSLRAGVMRIFKRTPFYVEEXVDGYNVRVALVS | | |
| DRVLAITRGGFVCPFTTERILDFVPEEFFKDYPHLVLVGEMAGPESPYLVEG | | |
| PPYVEEDIRFFLFDIQEKGTGKSLPVQERLKLAEEYGIPHVKVFGLYTVDRI | | |
| EDLYDLIERLSREGREGVVMKSPDMKRVVKYVTPFANVNDVKIGAKVFFELP | | |
| PGYFMSRIMRLAFYVAERRIKGERFEELARNLGKALLEPFVESIWDVEQGDE | | |
| IAEVFRIRVKRIETAYKMVTHFERLGLNIKIEDIEEVGGMWRITFKRAYDEA | | |
| TREIRELIGGRAFVD | | |
| Where X is any amino acid except K. | | |
| MVSSKFKDILYRLGIPEGKVEDLEARGGLVEDKFDDIKYLRIRNSVGKLRRG | 3 | Pyrococcus horikoshii |
| TVVLNDKFIILGFPHIKRIVNLKNGIKRTFKRGEFYVEEXVDGYNVRVVKFR | | |
| GKXLGITRGGFICPFTTERISDFIPEEFFKDHPNLILVGEMAGPESPYLVEG | | |
| PPYVKEDIQFFLFDIQELGTGRSLPVEERLKIAEEYGISHVEVFGKFTYKDL | | |
| EEIYEIVERLSREGREGIVMKSPDMRKMVKYVTPYANINDIKIGARVFYELP | | |
| PGYFTSRISRLAFYIAEKRLRGENFEELAKELGKALLQPLVESIHDVEQEDE | | |
| IAEVFKVRVKKIETAYKMVTHFEKLGLRIEIVDIEEMKGGWRITFKRLYPDA | | |
| TEEIRELIGGKSFVD | | |
| Where X is any amino acid except K. | | |
| MKEVVSSVYKEILVKLGLTEDRIETLEMKGGIIEDEFDGIRYVRFKDSAGKL | 4 | Pyrococcus abyssi |
| RRGTVVIDEEYVIPGFPHIKRIINLRSGIRRIFKRGEFYVEEXVDGYNVRVV | | |
| MYKGKMLGITRGGFICPFTTERIPDFVPQEFFKDNPNLILVGEMAGPESPYL | | |
| VEGPPYVKEDIQFFLFDVQEIKTGRSLPVEERLKIAEEYGINHVEVFGKYTK | | |
| DDVDELYQLIERLSKEGREGIIMKSPDMKKIVKYVTPYANINDIKIGARVFY | | |
| ELPPGYFTSRISRLAFYLAEKRIKGEEFERVAKELGSALLQPFVESIFDVEQ | | |
| EEDIHELFKVRVKRIETAYKMVTHFEKLGLKIEIVDIEEIKDGWRITFKRLY | | |
| PDATNEIRELIGGKAFVD | | |
| Where X is any amino acid except K. | | |
| MENMVSSKFKELLYTLGIPEDKVEILEARGGIMEDEFEGIRYLRFKNSVGKL | 5 | Pyrococcus furiosus |
| RRGTVLFEDGTTVFGFPHIKRIVNLSAGVRKIFKSSEFYVEEXVDGYNVRVV | | |
| KFKDRILGITRGGFICPYTTERIAEFVPEEFFKDHKDLVLVGEMAGPESPYL | | |
| VEGPPYVKEDIQFFLFDIQDIKTGSSLPVEERLKLAEEYGINHVEVFGRYSY | | |
| KDIDDLYELIERLSREGREGIVMKSPDMKKIVKYVTPYANINDIKIGARVFY | | |
| ELPGGYFTSRISRLAFYIAEKKIRGEELHNLALQLGKALLQPLVEAIHDVTQ | | |
| GDVIAERFRVRVRKIETAYKMVTHFEKLGLEIEIEDIEEIEGGWRVTFKRVY | | |
| PEATREIRDLIGGKAFVD | | |
| Where X is any amino acid except K. | | |
| MTWIKNPEPWMVNLVAEKLGLDVERVETLARHGTIRFRGYRDVVYALLRREI | 6 | Hyperthermus |
| AGHPEGTVVLLERNGARLVPGYPPIQRMVLPTIALPRHFIDKVVVEEXMNGY | | butylicus |
| NVRLVMFHRKLLAVTRGGFICPYTTARLERLIGGRVRELFREIDPETYTIAG | | |
| EVVGLENPYTRYFYPEAPRFDYFVFDLFHELKPLPPLERNELLEKYGLKHVR | | |
| LLGVIDKNDVEMFKQIVAELDREGREGVVAKDPEYRVPPLKYTTSAMNIGDV | | |
| RYGMRFFMEEGRSFLFSRLLRELFRAYEEGFGDAQLEKLALEFGRAATEPAL | | |
| ESIRKVAMGDMLYEEFELVFADEVELEEFTSYMAELGVDIVVVSTSREDEGL | | |
| RARMRKIKDTWIQLRKVLDTGLSPVD | | |
| Where X is any amino acid except K. | | |
| MTWIKNPEPWMVNLVAEKLGLDVERVETLARHGTIRFRGYRDVVYALLRREI | 7 | HbuRN12K106A |
| AGHPEGTVVLLERNGARLVPGYPPIQRMVLPTIALPRHFIDKVVVEEAMNGY | | |
| NVRLVMFHRKLLAVTRGGFICPYTTARLERLIGGRVRELFREIDPETYTIAG | | |
| EVVGLENPYTRYFYPEAPRFDYFVFDLFHELKPLPPLERNELLEKYGLKHVR | | |
| LLGVIDKNDVEMFKQIVAELDREGREGVVAKDPEYRVPPLKYTTSAVNIGDV | | |
| RYGMRFFMEEGRSFLFSRLLRELFRAYEEGFGDAQLEKLALEFGRAATEPAL | | |
| ESIRKVAMGDMLYEEFELVFADEVELEEFTSYMAELGVDIVVVSTSREDEGL | | |
| RARMRKIKDTWIQLRKVLDTGLSPVD | | |
| MASAAEVLASALRAVGVDPGSVDLEALSTRRSVRVSRFEDVVYVGFRRQFRG | 8 | Aeropyrum pernix |
| VPEGTLVAFRRGEQIVVWGYPSIKRMLLPRVAVPRWFPGPTVLVEEXMNGYN | | |
| VRVFTLGGMVYAATRGGLICPYTTRRLRRLYGGALQKILEDLGAEGSFIAGE | | |
| VVGLENPYTRYYYEEAPGFGYFIFDIFKGGRQLPPRVKFSLAPEYGLKTVNL | | |
| LAEIPATASGVERLYTIVEDLEKRGREGVIVKDPEGRVEPLKYTTSRTNIGD | | |
| IRLGMRYPFEEGRSFLFPRILREIFREWETGRRRYGELGEAILAPAIEAVEA | | |
| VSRGGRLVEEFELVFANEVEAEEVIAYFASLGVHLEIAGVARGVDGVRVAFR | | |
| KPRKSEGEIARILETGISPLD | | |
| Where X is any amino acid except K. | | |
| MDENELVNKLSDALGIEYEKLSKHIGRSIRLMKYGELNYVVFRRDLLGYREG | 9 | Staphylothermus |
| TTILLGEEPLIVHGYPSIQRLAFIEGVSKHMIDNVVVEEXMNGYNVRVVYYM | | marinus |
| NNIYAITRGGYICPYTTARIRKLYSKNIKLAYQEYPDTILVGEVVGTENPYV | | |
| IYDYPEARGFDYFIFDTMKKDKLQPLRIRDEIAEKYSLKTVRILDIINKRDI | | |
| DRLKTIINRLEKERREGVVLKDPYQRVPPLKYTTIYINIRDIWEGMRYPFDE | | |
| GRGYLFSRIVRLIAQGYEYDWNNTELDRIALKLGRAILEPAINSLKKRANGE | | |
| IIASKYTLVFPSEDDLSKYIEYAESIGMDFIFRVVEKREDGCIVVELFKMKE | | |
| THNIYTKMLKTGYSPLD | | |
| Where X is any amino acid except K. | | |
| MIRIPLERWMIEKLAEALNVNIEEAERLARRRNVVRLMKWRNVTYFSLRKDV | 10 | Pyrolobus fumarii |
| YGLREGTLIAVWPDGYRVVPGYPSIQRVLLPSVALPKHFIDKIVVEEXLNGY | | |
| NVRVVKLRDEIVAVTRGGLICPYTTQRIRKLYGDKLTSLFREEGEELVVAGE | | |
| VIGLENPYVRFYYPEAGGFAYFIFDIVHGEKFLPPHERKEIVEKHGLLHVPV | | |
| LGEIDKNDIKAFRKIIEDLERRGREGVVLKDPEYRVPPLKYTTSFINIHDIE | | |
| IGMRFPFDEGRNYLFSRILREIFKAVEEGWDDRRLLLAEQNLGKAILEPAIE | | |
| AVKEVKNGKMLYEEFMLPFDTRDDFEEFLDYMASLGVDIIVAGVEQRSDGSI | | |
| VARIRKVKDTWREVQKILETGLSPID | | |
| Where X is any amino acid except K. | | |
| MISPELVKEALKKKKVRSEEAFGLEYLRFNDDYKDIPRGTAIFKDFIIWGYP | 11 | Aquifex aeolicus |
| HIGRIFLLETGLREQFEAPFWVEEXVDGYNTRIFKYGDNYYALSRGGFICPF | | |
| TTDRLPDLIDLRILDENPDLVICAEVAGPENPYIEESPPYVKEDVQLFVFDF | | |
| MKKNEQGFLSQEEKMELIEKYNLPHVEILGRFTASEEGIKKIKEILKRFNEE | | |
| GREGVVFKEDSERNKRAKYITSYANLMDIKTNAKNMLQLPPEYYTNRILRLV | | |
| LFMYEEGLKTTEHLYEELGRAFIDGLFQAIEQFEKEHKVYKTFTCKFRKKEN | | |
| AIALLELLSKTSKHIQVKERRLEKEGDYWRLEFDKVFLNMTGLLGHLLSGGI | | |
| VYD | | |
| Where X is any amino acid except K. | | |

In some embodiments, the sequences in Table 1 above or Table 2 below are used to perform a sequence search (e.g., using BLAST) to find other hyper-thermostable ssRNA ligases from other species (e.g., by finding those with 30% . . . 50% . . . 60% or more homology). Once such ligases are identified, Motif I in such sequences can be located, and the lysine (K) in such Motif I can be mutated to another amino acid, preferably an alanine (A), serine (S), cysteine (C), valine (V), threonine (T), and Glycine (G). Such candidate enzymes can then be screened for ssDNA and ssRNA activities using (and thermostability), for example, using the same procedure as in Example 1 below. In this regard finding and designing hyper-thermostable lysine-mutant ssDNA/RNA ligases may be readily accomplished.

In certain embodiments, the hyper-thermostable lysine-mutant ssDNA/RNA ligases are used in sequencing methods, such as in attaching adapters to library fragments for subsequent sequencing. For example, in some embodiments, the disclosure provided herein finds use in a Second Generation (a.k.a. Next Generation or Next-Gen), Third Generation (a.k.a. Next-Next-Gen), or Fourth Generation (a.k.a. N3-Gen) sequencing technology including, but not limited to, pyrosequencing, sequencing-by-ligation, single molecule sequencing, sequence-by-synthesis (SBS), semiconductor sequencing, massive parallel clonal, massive parallel single molecule SBS, massive parallel single molecule real-time, massive parallel single molecule real-time nanopore technology, etc. Morozova and Marra provide a review of some such technologies in Genomics, 92: 255 (2008), herein incorporated by reference in its entirety.

Any number of DNA sequencing techniques are suitable, including fluorescence-based sequencing methodologies (See, e.g., Birren et al., Genome Analysis: Analyzing DNA, 1, Cold Spring Harbor, N.Y.; herein incorporated by reference in its entirety). In some embodiments, the present disclosure finds use in automated sequencing techniques understood in that art. In some embodiments, the present technology finds use in parallel sequencing of partitioned amplicons (PCT Publication No: WO2006084132, herein incorporated by reference in its entirety). In some embodiments, the technology finds use in DNA sequencing by parallel oligonucleotide extension (See, e.g., U.S. Pat. No. 5,750,341, and U.S. Pat. No. 6,306,597, both of which are herein incorporated by reference in their entireties). Additional examples of sequencing techniques in which the technology finds use include the Church polony technology (Mitra et al., 2003, Analytical Biochemistry 320, 55-65; Shendure et al., 2005 Science 309, 1728-1732; U.S. Pat. No. 6,432,360, U.S. Pat. No. 6,485,944, U.S. Pat. No. 6,511,803; all of which are herein incorporated by reference in their entireties), the 454 picotiter pyrosequencing technology (Margulies et al., 2005 Nature 437, 376-380; US 20050130173; herein incorporated by reference in their entireties), the Solexa single base addition technology (Bennett et al., 2005, Pharmacogenomics, 6, 373-382; U.S. Pat. No. 6,787,308; U.S. Pat. No. 6,833,246; herein incorporated by reference in their entireties), the Lynx massively parallel signature sequencing technology (Brenner et al. (2000). Nat. Biotechnol. 18:630-634; U.S. Pat. No. 5,695,934; U.S. Pat. No. 5,714,330; all of which are herein incorporated by reference in their entireties), and the Adessi PCR colony technology (Adessi et al. (2000). Nucleic Acid Res. 28, E87; WO 00018957; herein incorporated by reference in its entirety).

Next-generation sequencing (NGS) methods share the common feature of massively parallel, high-throughput strategies, with the goal of lower costs in comparison to older sequencing methods (see, e.g., Voelkerding et al., Clinical Chem., 55: 641-658, 2009; MacLean et al., Nature Rev. Microbiol., 7: 287-296; each herein incorporated by reference in their entirety). NGS methods can be broadly divided into those that typically use template amplification and those that do not. Amplification-requiring methods include pyrosequencing commercialized by Roche as the 454 technology platforms (e.g., GS 20 and GS FLX), Life Technologies/Ion Torrent, the Solexa platform commercialized by Illumina, GnuBio, and the Supported Oligonucleotide Ligation and Detection (SOLiD) platform commercialized by Applied Biosystems. Non-amplification approaches, also known as single-molecule sequencing, are exemplified by the HeliScope platform commercialized by Helicos BioSciences, and emerging platforms commercialized by VisiGen, Oxford Nanopore Technologies Ltd., and Pacific Biosciences, respectively.

In pyrosequencing (Voelkerding et al., Clinical Chem., 55: 641-658, 2009; MacLean et al., Nature Rev. Microbiol., 7: 287-296; U.S. Pat. No. 6,210,891; U.S. Pat. No. 6,258,568; each herein incorporated by reference in its entirety), template DNA is fragmented, end-repaired, ligated to adapters, and clonally amplified in-situ by capturing single template molecules with beads bearing oligonucleotides complementary to the adaptors. Each bead bearing a single template type is compartmentalized into a water-in-oil microvesicle, and the template is clonally amplified using a technique referred to as emulsion PCR. The emulsion is disrupted after amplification and beads are deposited into individual wells of a picotitre plate functioning as a flow cell during the sequencing reactions. Ordered, iterative introduction of each of the four dNTP reagents occurs in the flow cell in the presence of sequencing enzymes and luminescent reporter such as luciferase. In the event that an appropriate dNTP is added to the 3′ end of the sequencing primer, the resulting production of ATP causes a burst of luminescence within the well, which is recorded using a CCD camera. It is possible to achieve read lengths greater than or equal to 400 bases, and 10⁶ sequence reads can be achieved, resulting in up to 500 million base pairs (Mb) of sequence.

In the Solexa/Illumina platform (Voelkerding et al., Clinical Chem., 55: 641-658, 2009; MacLean et al., Nature Rev. Microbiol., 7: 287-296; U.S. Pat. No. 6,833,246; U.S. Pat. No. 7,115,400; U.S. Pat. No. 6,969,488; each herein incorporated by reference in its entirety), sequencing data are produced in the form of shorter-length reads. In this method, single-stranded fragmented DNA is end-repaired to generate 5′-phosphorylated blunt ends, followed by Klenow-mediated addition of a single A base to the 3′ end of the fragments. A-addition facilitates addition of T-overhang adaptor oligonucleotides, which are subsequently used to capture the template-adaptor molecules on the surface of a flow cell that is studded with oligonucleotide anchors. The anchor is used as a PCR primer, but because of the length of the template and its proximity to other nearby anchor oligonucleotides, extension by PCR results in the “arching over” of the molecule to hybridize with an adjacent anchor oligonucleotide to form a bridge structure on the surface of the flow cell. These loops of DNA are denatured and cleaved. Forward strands are then sequenced with reversible dye terminators. The sequence of incorporated nucleotides is determined by detection of post-incorporation fluorescence, with each fluor and block removed prior to the next cycle of dNTP addition. Sequence read length ranges from 36 nucleotides to over 250 nucleotides, with overall output exceeding 1 billion nucleotide pairs per analytical run.

Sequencing nucleic acid molecules using SOLiD technology (Voelkerding et al., Clinical Chem., 55: 641-658, 2009; MacLean et al., Nature Rev. Microbiol., 7: 287-296; U.S. Pat. No. 5,912,148; U.S. Pat. No. 6,130,073; each herein incorporated by reference in their entirety) also involves fragmentation of the template, ligation to oligonucleotide adapters, attachment to beads, and clonal amplification by emulsion PCR. Following this, beads bearing template are immobilized on a derivatized surface of a glass flow-cell, and a primer complementary to the adaptor oligonucleotide is annealed. However, rather than utilizing this primer for 3′ extension, it is instead used to provide a 5′ phosphate group for ligation to interrogation probes containing two probe-specific bases followed by 6 degenerate bases and one of four fluorescent labels. In the SOLiD system, interrogation probes have 16 possible combinations of the two bases at the 3′ end of each probe, and one of four fluors at the 5′ end. Fluor color, and thus identity of each probe, corresponds to specific color-space coding schemes. Multiple rounds (usually 7) of probe annealing, ligation, and fluor detection are followed by denaturation, and then a second round of sequencing using a primer that is offset by one base relative to the initial primer. In this manner, the template sequence can be computationally re-constructed, and template bases are interrogated twice, resulting in increased accuracy. Sequence read length averages 35 nucleotides, and overall output exceeds 4 billion bases per sequencing run.

In certain embodiments, the technology described herein finds use in nanopore sequencing (see, e.g., Astier et al., J. Am. Chem. Soc. 2006 Feb. 8; 128(5):1705-10, herein incorporated by reference). The theory behind nanopore sequencing has to do with what occurs when a nanopore is immersed in a conducting fluid and a potential (voltage) is applied across it. Under these conditions a slight electric current due to conduction of ions through the nanopore can be observed, and the amount of current is exceedingly sensitive to the size of the nanopore. As each base of a nucleic acid passes through the nanopore, this causes a change in the magnitude of the current through the nanopore that is distinct for each of the four bases, thereby allowing the sequence of the DNA molecule to be determined.

In certain embodiments, the technology described herein finds use in HeliScope by Helicos BioSciences (Voelkerding et al., Clinical Chem., 55: 641-658, 2009; MacLean et al., Nature Rev. Microbiol., 7: 287-296; U.S. Pat. No. 7,169,560; U.S. Pat. No. 7,282,337; U.S. Pat. No. 7,482,120; U.S. Pat. No. 7,501,245; U.S. Pat. No. 6,818,395; U.S. Pat. No. 6,911,345; U.S. Pat. No. 7,501,245; each herein incorporated by reference in their entirety). Template DNA is fragmented and polyadenylated at the 3′ end, with the final adenosine bearing a fluorescent label. Denatured polyadenylated template fragments are ligated to poly(dT) oligonucleotides on the surface of a flow cell. Initial physical locations of captured template molecules are recorded by a CCD camera, and then label is cleaved and washed away. Sequencing is achieved by addition of polymerase and serial addition of fluorescently-labeled dNTP reagents. Incorporation events result in fluor signal corresponding to the dNTP, and signal is captured by a CCD camera before each round of dNTP addition. Sequence read length ranges from 25-50 nucleotides, with overall output exceeding 1 billion nucleotide pairs per analytical run.

The Ion Torrent technology is a method of DNA sequencing based on the detection of hydrogen ions that are released during the polymerization of DNA (see, e.g., Science 327(5970): 1190 (2010); U.S. Pat. Appl. Pub. Nos. 20090026082, 20090127589, 20100301398, 20100197507, 20100188073, and 20100137143, incorporated by reference in their entireties for all purposes). A microwell contains a template DNA strand to be sequenced. Beneath the layer of microwells is a hypersensitive ISFET ion sensor. All layers are contained within a CMOS semiconductor chip, similar to that used in the electronics industry. When a dNTP is incorporated into the growing complementary strand a hydrogen ion is released, which triggers a hypersensitive ion sensor. If homopolymer repeats are present in the template sequence, multiple dNTP molecules will be incorporated in a single cycle. This leads to a corresponding number of released hydrogens and a proportionally higher electronic signal. This technology differs from other sequencing technologies in that no modified nucleotides or optics is used. The per-base accuracy of the Ion Torrent sequencer is ˜99.6% for 50 base reads, with ˜100 Mb to 100 Gb generated per run. The read-length is 100-300 base pairs. The accuracy for homopolymer repeats of 5 repeats in length is ˜98%. The benefits of ion semiconductor sequencing are rapid sequencing speed and low upfront and operating costs.

The technology disclosed herein finds use in another nucleic acid sequencing approach developed by Stratos Genomics, Inc. and involves the use of Xpandomers. This sequencing process typically includes providing a daughter strand produced by a template-directed synthesis. The daughter strand generally includes a plurality of subunits coupled in a sequence corresponding to a contiguous nucleotide sequence of all or a portion of a target nucleic acid in which the individual subunits comprise a tether, at least one probe or nucleobase residue, and at least one selectively cleavable bond. The selectively cleavable bond(s) is/are cleaved to yield an Xpandomer of a length longer than the plurality of the subunits of the daughter strand. The Xpandomer typically includes the tethers and reporter elements for parsing genetic information in a sequence corresponding to the contiguous nucleotide sequence of all or a portion of the target nucleic acid. Reporter elements of the Xpandomer are then detected. Additional details relating to Xpandomer-based approaches are described in, for example, U.S. Pat. Pub No. 20090035777, which is incorporated herein in its entirety.

Other single molecule sequencing methods include real-time sequencing by synthesis using a VisiGen platform (Voelkerding et al., Clinical Chem., 55: 641-58, 2009; U.S. Pat.

No. 7,329,492; U.S. patent application Ser. No. 11/671956; U.S. patent application Ser. No. 11/781166; each herein incorporated by reference in their entirety) in which immobilized, primed DNA template is subjected to strand extension using a fluorescently-modified polymerase and florescent acceptor molecules, resulting in detectable fluorescence resonance energy transfer (FRET) upon nucleotide addition.

In certain embodiments, provided herein are compositions, kits, and systems comprising a hyper-thermostable lysine-mutant ssDNA/RNA ligase encoded by SEQ ID NOs:1-11, or encoded by a sequence with substantial identity with SEQ ID NOs:1-11. As applied to such polypeptides, the term “substantial identity” means that two peptide sequences, when optimally aligned, such as by the programs GAP or BESTFIT using default gap weights, share at least 80 percent sequence identity, or at least 90 percent sequence identity, or at least 95 percent sequence identity or more (e.g., 95% . . . 97% . . . or 99% percent sequence identity). In particular embodiments, residue positions which are not identical differ by conservative amino acid substitutions. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. In some embodiments, the conservative amino acids substitution groups are: valine-leucine-isoleucine, phenylalanine-tyrosine, lysine-arginine, alanine-valine, and asparagine-glutamine. In certain embodiments, provided herein are peptides that have substantial identity to at least a portion of the amino acid sequences shown in SEQ ID NOS: 1-11.

### Experimental

### EXAMPLE 1

### Mutant Hyper-Thermostable ssDNA/RNA Ligases

First, a series of hyper-thermostable RNA ligases were identified in a search of the art. This list, in Table 2 below, includes hyperthermophilic archaea species, some of which can endure environments with over 100° C.

TABLE 2

| | | | Optimal | IDs |
|---|---|---|---|---|
| | | Growth | growth | used in |
| | | tempera- | temper- | this ap- |
| | | ture range | ature | plica- |
| Genbank ID | Species name | (° C.) | (° C.) | tion |
| WP_011250496.1 | Thermococcus | 65-100 | 85 | TkoRnl2 |
| | kodakarensis | | | |
| WP_013906275.1 | Pyrococcus | 80-108 | 98 | PyaRnl2 |
| | yayanosii | | | |
| WP_010884602.1 | Pyrococcus | 88-104 | 98 | PhoRnl2 |
| | horikoshii | | | |
| WP_048147341.1 | Pyrococcus | 90-102 | 96 | Pab1020 |
| | abyssi* | | | |
| WP_014835129.1 | Pyrococcus | 70-103 | 100 | PfuRnl2 |
| | furiosus | | | |
| WP_011822689.1 | Hyperthermus | Up to 112 | 95-107 | HbuRnl2 |
| | butylicus | | | |
| BAA80567.2 | Aeropyrum | 70-100 | 90-95 | AperRnl2 |
| | pernix | | | |
| WP_011839720.1 | Staphylothermus | Up to 98 | 85-92 | SmarRnl2 |
| | marinus | | | |
| WP_014027000.1 | Pyrolobus fumarii | Up to 113 | | PfumRnl2 |
| WP_010880692.1 | Aquifex aeolicus | 85-95 | | AaeRnl2 |

*Pab1020 has been previously studied and shown to have ssRNA ligation activity, but no ssDNA ligation activity (Brooks et al, Protein Science, 2008).

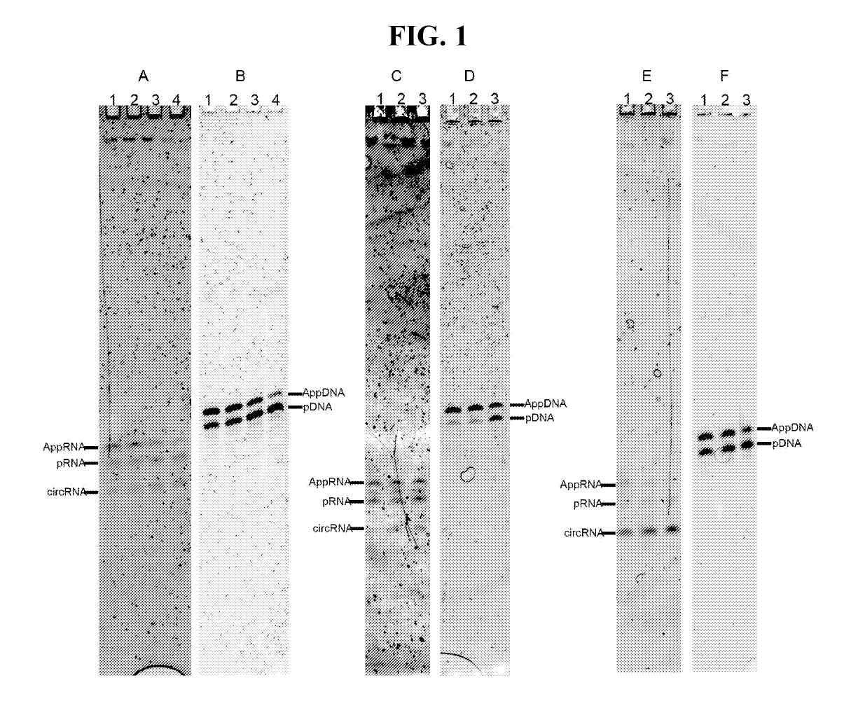

Three of the 10 ligases from Table 2 (PhoRnl2, PfuRnl2, HbuRnl2) were synthesized using optimized E. coli codon, expressed and purified, and were examined for their activity on single-strand RNA and DNA substrate at high temperature (90° C. as in FIG. 1). As shown in FIG. 1, the enzymes' activities were assayed under a titration of ATP concentration using 5′-phosphorylated ssRNA and 5′-phosphorylated ssDNA oligonucleotides as substrates. As shown in FIGS. 1A, 1C and 1E, all enzymes tested can turn 5′-phosphorylated ssRNA to 5′-adenylated ssRNA and circularized ssRNA. Under higher concentration of ATP, the ligation reactions appear to stall after the adenyltransfer step (step 2), so that more adenylated products were accumulated. Due to the proximity of the ends in solution within the same ssRNA molecule, intra-molecule ligation is much more preferred than the inter-molecule ligation, so that the major ligation product is the circularized ssRNA. This evidence support that all enzymes are active hyperthermostable ssRNA ligases.

For activity on ssDNA, however, although all enzymes can turn 5′-phosphorylated ssDNA to 5′-adenylated ssDNA, as shown in FIGS. 1B, 1D and 1F, no circularized ssDNA is formed under varying concentration of ATP, suggesting that they do not possess ssDNA ligation activity, which is also consistent with the Pab1020 study (Brooks et al, Protein Science, 2008). Accumulation of 5′-adenylated ssDNA is higher under higher concentration of ATP, suggesting a similar stall after the step 2 of the adentltransfer. In the case of PfuRnl2, when the ATP concentration is higher than 10 uM, it is able to adenylate over 90% of the 5′-phosphorylated ends of ssDNA. The other two enzymes exhibit much lower adenylation efficiency under the same condition (FIG. 1). This unique property makes wild-type PfuRnl2 a useful hyperthermostable 5′-adenylase for ssRNA and ssDNA. While the present invention is not limited to any particular mechanism, and an understanding of the mechanism is not necessary to practice the invention, based on these results, it is believed that, although these enzymes can perform the step 2 of adenyltransfer on the 5′-phosphorylated ends of ssDNA, they cannot accomplish the final step of the ligation on ssDNA. In other words, they are not ssDNA ligases in nature.

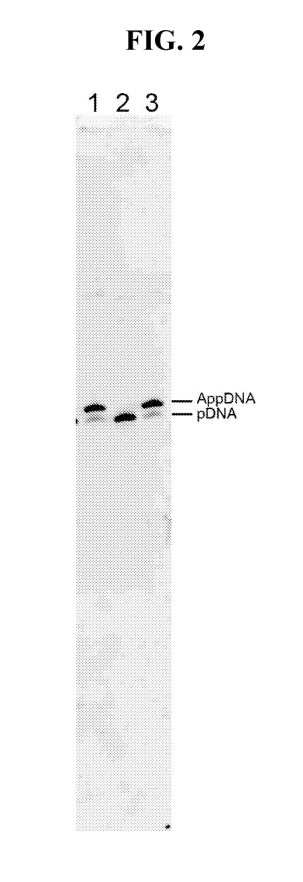

Next, one of the RNA ligases (HbuRnl2) was tested for its activity on a 5′-adenylated ssDNA under conditions with or without ATP. As shown in FIG. 2, wild-type HbuRnl2 can efficiently de-adenylate the 5′-adenylated ssDNA in the absence of ATP (lane 2). The de-adenylation is the reverse process of the step 2 in the ligation reaction, in which the un-adenylated enzyme transfers the AMP back to its catalytic lysine from the 5′-adenylated ends. In the presence of high concentration ATP, this reverse step 2 reaction does not occur (FIG. 2, lane 1). While the present invention is not limited to any particular mechanism, and an understanding of the mechanism is not necessary to practice the invention, by combining the observation that HbuRnl2, PfuRnl2, and PhoRnl2 can adenylate a varying proportion of 5′-phosphorylated ssDNA under a titration of ATP, it was hypothesized that the reverse step 2 reaction may block the ligation reaction from moving forward. Reducing the reverse step 2 reaction may push the reaction forward to finish the step 3 of the ligation on ssDNA.

In step 2 of the ligation, the adenyl group is transferred from the catalytic lysine in the conserved Motif I in ligases to the 5′-phosphorylated end. Catalytic lysine resides in a conserved motif EKx(D/N/H)G (SEQ ID NO:32). Thus, while the present invention is not limited to any particular mechanism, and an understanding of the mechanism is not necessary to practice the present invention, by mutating the catalytic lysine in Motif I to other amino acids, the step 2 reverse reaction may be blocked or reduced, so that the step 3 ligation, which is the joining between the 3′-OH end and the 5′-adenylated end, can occur.

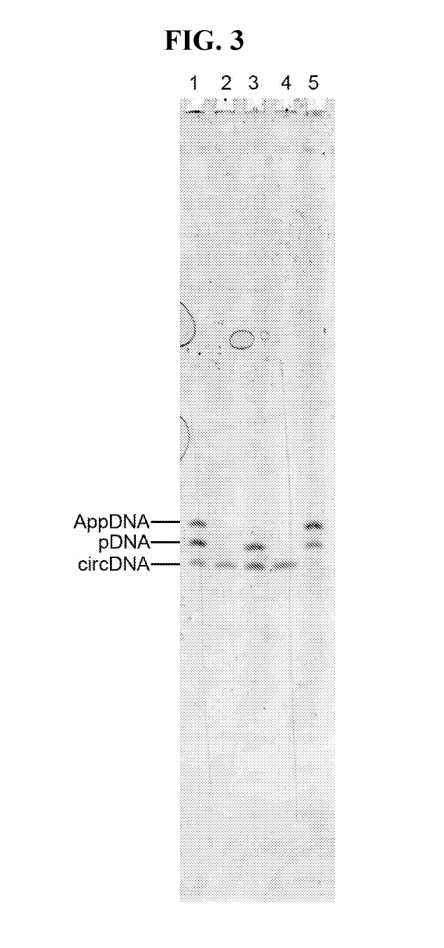

Next, HbuRnl2 was chosen as a target, and mutant HbuRnl2K106A (SEQ ID NO:7) was constructed and purified, and its activity was assayed in step 3 ligation. As shown in FIG. 3, HbuRnl2K106A can catalyze the intra-molecule ligation by turning 5′-adenylated ssDNA into circularized ssDNA. After reaction, the circularized ssDNA is resistant to Exonuclease I digestion and was retained on gel after Exol treatment (FIG. 3, lane 2). As a control and comparison, the ligation reaction catalyzed by the wild-type MthRnl at 65° C. was also ran and the results revealed the same circularized ssDNA (FIG. 3, lanes 3 and 4). Overall, these results confirm that although the wild-type HbuRnl2 does not have the step 3 ligation activity of ssDNA, by mutating the catalytic lysine in HbuRnl2, the enzyme acquires a new activity of ligating 5′-adenylated end and 3′-OH end of ssDNA.

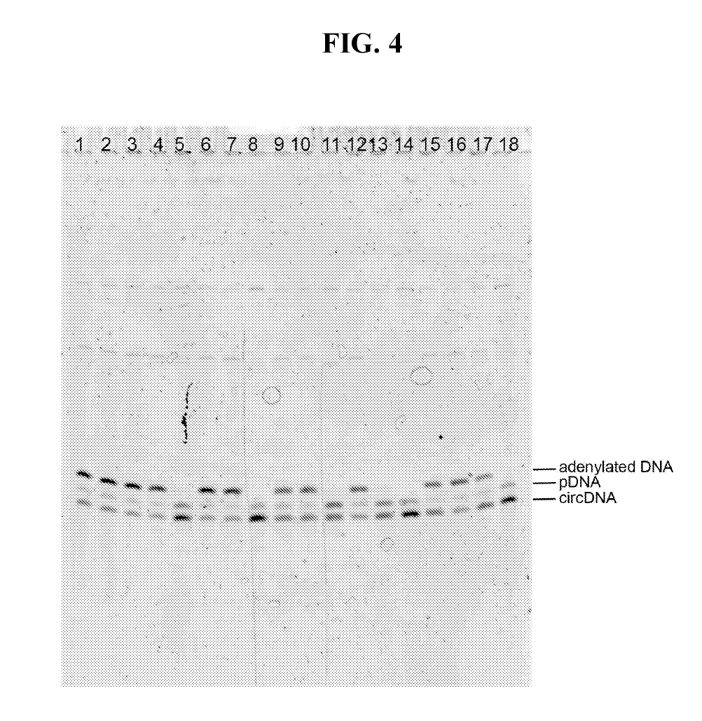

Next, lysine 106 in HbuRnl2 was changed to other amino acids, and mutants were assayed for its adenylated ssDNA circularization activity. As shown in FIG. 4, mutants K106T, K106G, K106V, K106C, K106S are active in adenylated ssDNA circularization as well as K106A. Other lysine mutants are inactive in adenylated ssDNA circularization.

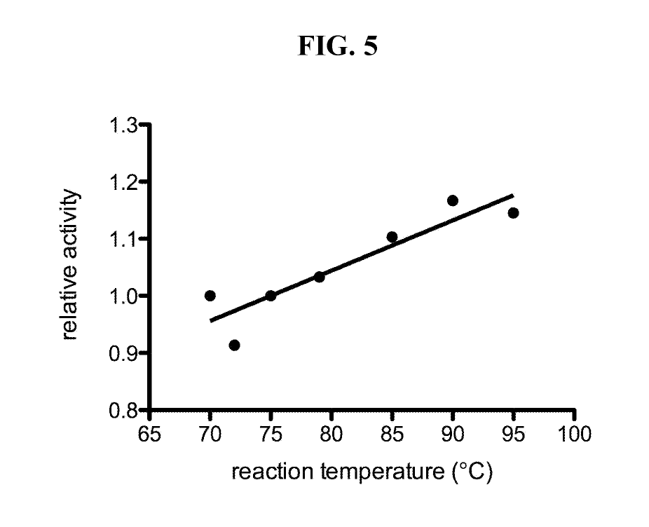

The optimal reaction temperature range for HbuRnl2K106A was then examined. As shown in FIG. 5, the 5′-adenylated ssDNA circularization activity was assayed in a range of temperature from 70 to 95° C. The activity appears to increase moderately from 70 to 90° C., and reach a plateau after 90° C. For this reason, biochemical assays were conducted at 90° C. This observation is also consistent with the enzyme's origin from a hyperthemophilic archaea and indicates that mutation does not alter its hyperthermostability.

Due to the intrinsic flexibility of ssDNA/RNA and the proximity of the ends of the same oligonucleotide, intra-molecule circularization by ligation occurs in a much higher frequency than inter-molecule concatenation. This may be part of the reason that TS2126 ligase was named “CircLigase” (WO2004/027056A2). Circularization of ssDNA/RNA can be useful for a lot of applications. For example, the circularized oligonucleotides are resistant to exonuclease digestion and can serve as templates for amplification, such as rolling circle amplification. Oftentimes, however, there is a desire to promote inter-molecule ssDNA/RNA ligation instead of intra-molecule circularization, for example, when one wants to attach an oligonucleotide adaptor to a library of ssDNA/RNA.

A number of strategies can be used to promote inter-molecule ligation between ssDNA/RNA. For example, 3′-OH ends of ssDNA/RNA can be modified, such as by using NH₂ group or many other modification groups, so that the OH group is not present for the intra-molecule ligation to occur. In addition, Mn²⁺ and ligation enhancer, such as PEG8000, may be used (Zhelkovsky A., et al, BMC Biotechnology, 2012, herein incorporated by reference).

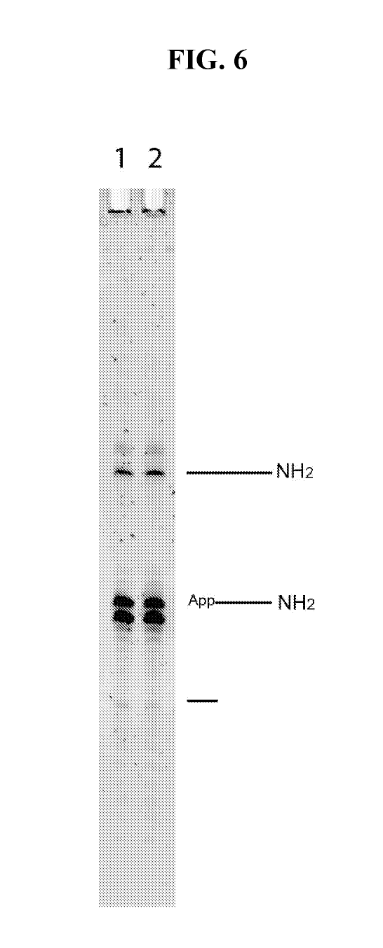

In FIG. 6, it was shown that by using a molar excess of 5′-adenylated, 3′-NH₂ modified adaptor, inter-molecule ligation is not only possible, but also can approach efficiently to >90% completion for HbuRnl2K106A. Such property is useful, for example, in adaptor ligation in many applications.

Per the above, it was determined that mutant HbuRnl2K106A can catalyze the ligation between two pieces of ssRNA, in which both ligation donor and acceptor are RNA, and it can catalyze the ligation between two pieces of ssDNA, in which both ligation donor and acceptor are DNA. It is further demonstrated that it can ligate between ssRNA donor and ssDNA acceptor, or between ssDNA donor and ssRNA acceptor (data not shown). These properties are useful for many applications. For example, current protocols for small RNA cloning and sequencing uses T4Rnl2 and its mutants at 37° C. or lower for the attachment of 5′-adenylated ssDNA adaptor to the 3′-OH of ssRNA. One could use HbuRnl2K106A for the same purpose but at a much higher temperature with the advantage of minimizing secondary structure.

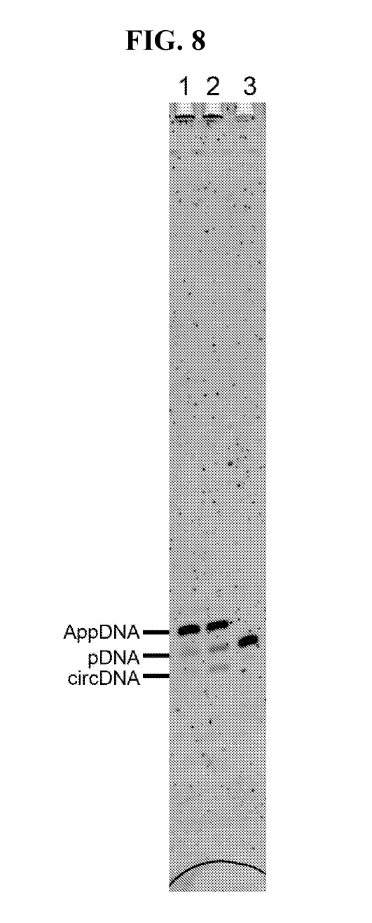

Per the above, it was shown that PfuRnl2 can efficiently adenylate 5′-phosphorylated ends of ssDNA (FIG. 1). As shown in FIG. 8, one can also mix PfuRnl2 and HbuRnl2K106A in one-pot reaction so that PfuRnl2 can adenylate the 5′-phosphorylated ends and HbuRnl2K106A can join the 5′-adenylated ends with 3′-OH ends of ssDNA. Thus, the enzyme mixture can perform step 1 to step 3 of the ligation reaction with a high reaction temperature.

### Methods and Materials for Example 1

### 1. Synthesis, Mutagenesis, Expression and Purification of Hyperthermostable Single-Stranded RNA/DNA Ligases

All genes are synthesized as double-stranded DNA fragment with optimized E. coli codon usage and 6X-His-tag at the N-terminus by IDT (Integrated DNA Technologies, Coralville, Iowa). The DNA was then inserted into expression vectors pTXB1 under the control of T7 promoter using Gibson Assembly (NEB #E2611). Expression constructs were transformed into T7 express E. coli strain (NEB #C2566). For expression in culture, the cells were grown in LB media to OD ˜0.6, upon which a final concentration of 0.5 mM IPTG was added. The induced cultures were kept grown by shaking at 20° C. for overnight.

For purification, 250 ml of induced cell culture was first pelleted by centrifugation. The cell pellet was then re-suspended in buffer containing 20 mM Tris (pH=7.5), 150 mM NaCl, 1× FastBreak (Promega), 200 ug/ml lysozyme, and sonicated. Cell lysates were then centrifuged at 15,000 g for 20 min at 4° C. The cleared lysate is then purified successively on a Ni-NTA column and a Heparin column.

Site-directed mutagenesis (e.g., by using New England Biolabs' Q5 mutagenesis kit) was performed with the cloned constructs to introduce mutation to the conserved lysine residue.

### EXAMPLE 2

### Single Stranded Adapter Ligation

Hyperthermostable ssDNA/RNA ligases (e.g., as described in SEQ ID NOS:1-11) can be used in the library preparation process for next-generation high-throughput sequencing. Currently, the dominant library construction methods include a ligation step where double-stranded library fragments are ligated to double-stranded adaptors. Single-strand ligation based method exists, but by using the CircLigase with a optimal reaction temperature around 65° C. (Gansauge M T, Nat. Protol., 2013).

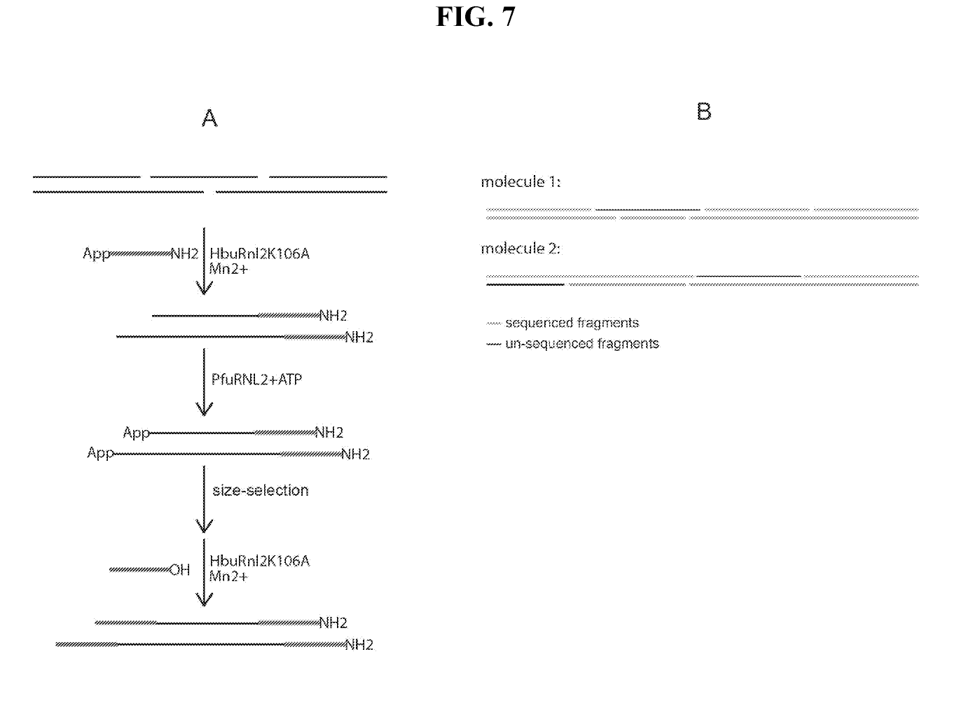

As illustrated in FIG. 7A, large DNA is first fragmented, for example, by using enzymatic method or by mechanic/ultrasound shearing. Depending on the fragmentation method, the ends of the fragments may require a repairing step using, for example, T4 polynucleotide kinase. The fragmented DNA is then ligated to the first single-strand DNA adaptor, with 5′-adenylated end and 3′-NH₂ end. This inter-molecule ligation is catalyzed by one of the ssDNA/RNA ligases disclosed herein, such as HbuRnl2K106A, which may be in the presence of Mn2+, and PEG8000 at high temperature.

After ligation, a cleanup, e.g., by using AMPure beads or other methods well known in the art, is used to recover all fragments ligated with the first adaptor. The recovered fragments are then adenylated, for example, by using the hyperthermostable 5′-adenylase PfuRnl2 in high concentration of ATP. Then, the 5′-adenylated DNA fragments are ligated with the second adaptor, for example, with regular 5′-OH and 3′-OH ends. This ligation reaction is catalyzed by one of the mutant ligases herein, such as the hyperthermostable HbuRnl2K106A. After ligation, the ligated DNA fragments are purified and ready for further downstream steps, such as via polymerase chain reaction (PCR) or cluster generation.

A number of advantages for a single-strand ligation based library construction exist with the presently disclosed methods, compositions, and systems. First, workflow of NGS sample preparation can be simplified, without end polishing steps. Second, compared with previous protocols, much less bias may exist, especially for long stretches of oligonucleotides in the library, due to high reaction temperature. Third, sequenced reads may be concatenated to form synthetic long reads and assigned to different starting genomic DNA molecules, with the assumption of random breakpoints introduced by the shearing process and relatively deep sequencing coverage so that most of the ssDNA fragments are sequenced (FIG. 7B).

### REFERENCES

Blondal et al., Isolation and characterization of a thermostable RNA ligase 1 from a Thermus scotoductus bacteriophage TS2126 with good single-stranded DNA ligation properties. Nucleic Acids Res. 2005 Jan. 7;33(1):135-42.

Blondal et al., Discovery and characterization of a thermostable bacteriophage RNA ligase homologous to T4 RNA ligase 1. Nucleic Acids Res. 2003, 31(24):7247-54.

Brooks et al., The structure of an archaeal homodimeric ligase which has RNA circularization activity. Protein Sci. 2008, 17(8):1336-45.

Gansauge and Meyer, Single-stranded DNA library preparation for the sequencing of ancient or damaged DNA. Nat Protoc. 2013, 8(4):737-48.

Ho and Shuman, Bacteriophage T4 RNA ligase 2 (gp24.1) exemplifies a family of RNA ligases found in all phylogenetic domains. Proc Natl Acad Sci U S A. 2002, 99(20):12709-14.

Lau et al., An abundant class of tiny RNAs with probable regulatory roles in Caenorhabditis elegans. Science. 2001, 294(5543):858-62.

Lehman I R, DNA ligase: structure, mechanism, and function. Science. 1974 Nov. 29;186(4166):790-7.

Lindahl and Barnes, Mammalian DNA ligases. Annu Rev Biochem. 1992;61:251-81.

Silber et al., Purification and properties of bacteriophage T4-induced RNA ligase, Proceedings of the National Academy of Sciences of the United States of America, vol. 69, no. 10, pp. 3009-3013, 1972.

Tomkinson et al., T. DNA ligases: structure, reaction mechanism, and function. Chem Rev. 2006, 106(2):687-99.

Torchia et al., Archaeal RNA ligase is a homodimeric protein that catalyzes intramolecular ligation of single-stranded RNA and DNA. Nucleic Acids Res. 2008, 36(19):6218-27.

US2014/0128292 A1 Methods for improving ligation steps to minimize bias during production of libraries for massively parallel sequencing

Viollet et al., T4 RNA ligase 2 truncated active site mutants: improved tools for RNA analysis. BMC Biotechnol. 2011, 11:72.

WO2004/027056A2 Methods of use for thermostable RNA ligases

Yin et al., Structure-function analysis of T4 RNA ligase 2. J Biol Chem. 2003, 278(20):17601-8

Zhuang et al., Structural bias in T4 RNA ligase-mediated 3′-adapter ligation. Nucleic Acids Res. 2012, 40(7):e54.

Zhelkovsky and McReynolds, Simple and efficient synthesis of 5′ pre-adenylated DNA using thermostable RNA ligase. Nucleic Acids Res. 2011, 39(17):e117.

Zhelkovsky and McReynolds, Structure-function analysis of Methanobacterium thermoautotrophicum RNA ligase—engineering a thermostable ATP independent enzyme. BMC Mol Biol. 2012, 13:24.

All publications and patents mentioned in the specification and/or listed below are herein incorporated by reference. Various modifications and variations of the described method and system of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the relevant fields are intended to be within the scope described herein.

We claim:

1. A method of ligating single-stranded nucleic acid comprising:
a) combining in a reaction mixture:
i) a by per-thermostable lysine-mutant ssDNA/RNA ligase which is a mutated version of a precursor hyper-thermostable ssRNA ligase,
wherein said precursor hyper-thermostable ssRNA ligase has a Motif I EKx(D/N/H)G (SEQ ID NO:32) and possess ssRNA ligase activity, but not ssDNA ligase activity, at a temperature of at least 75° C., and
wherein said hyper-thermostable lysine-mutant ssDNA/RNA ligase has an amino acid substitution at said K (lysine) in said Motif I, and possesses both ssRNA ligase and ssDNA ligase activity at a temperature of at least 75° C.,
ii) a first single stranded nucleic acid sequence with a 5′ end base that is adenylated, and
iii) a second single stranded nucleic acid sequence with a 3′ end base; and
b) incubating said reaction mixture at a temperature of at least 75° C. such that said hyper-thermostable lysine-mutant ssDNA/RNA ligase ligates said 5′ adenylated end of said first single stranded nucleic acid sequence to said 3′ end of said second single stranded nucleic acid sequence to form a ligated nucleic acid sequence.

2. The method of claim 1, wherein said 5′ end base of said first single stranded nucleic acid sequence is a DNA base, and wherein said 3′ end base of said second single stranded nucleic acid sequence is an RNA base or a DNA base.

3. The method of claim 1, wherein said 5′ end base of said first single stranded nucleic acid sequence is a DNA base or an RNA base, and wherein said 3′ end base of said second single stranded nucleic acid sequence is a DNA base.

4. The method of claim 1, wherein all of the bases in said first single stranded nucleic acid sequence are DNA bases.

5. The method of claim 1, wherein all of the bases in said second single stranded nucleic acid sequence are DNA bases.

6. The method of claim 1, wherein said amino acid substitution for said K (lysine) is an amino acid selected from the group consisting of: alanine (A), serine (S), cysteine (C), valine (V), threonine (T), and Glycine (G).

7. The method of claim 1, wherein said precursor hyper-thermostable ssRNA ligase is a wild-type hyper-thermostable ssRNA ligase.

8. The method of claim 7, wherein said wild-type hyper-thermostable ssRNA ligase is from a species selected from the group consisting of: Thermococcus kodakarensis, Pyrococcus, yayanosii, Pyrococcus horikoshii, Pyrococcus abyssi, Pyrococcus furiosus, Hyperthermus butylicus, Aeropyrum pernix, Staphylothermus marinus, Pyrolobus fumarii, and Aquifex aeolicus.

9. The method of claim 1, wherein said hyper-thermostable lysine-mutant ssDNA/RNA ligase is encoded by one of the amino acid sequences shown in SEQ ID NO:1-11.

10. The method of claim 1, wherein said hyper-thermostable lysine-mutant ssDNA/RNA ligase is encoded by an amino acid sequence that has at least 95% sequence identity to one of the amino acid sequences shown in SEQ ID NO:1-11.

11. The method of claim 1, wherein said incubating said reaction mixture is at a temperature of at least 85° C.

12. The method of claim 1, wherein said incubating said reaction mixture is at a temperature of at least 95° C.

13. The method of claim 1, wherein said first single stranded nucleic acid sequence is a sequencing adapter.

14. The method of claim 1, wherein said second single stranded nucleic acid sequence comprises a sequencing library fragment.

15. The method of claim 1, further comprising c) sequencing said ligated nucleic acid sequence.

16. A system or kit comprising:
a) a hyper-thermostable lysine-mutant ssDNA/RNA ligase which is a mutated version of a precursor hyper-thermostable ssRNA ligase,
wherein said precursor hyper-thermostable ssRNA ligase has a Motif I EKx(D/N/H)G (SEQ ID NO:32) and possess ssRNA ligase activity, but not ssDNA ligase activity, at a temperature of at least 75° C., and
wherein said hyper-thermostable lysine-mutant ssDNA/RNA ligase has an amino
acid substitution at said K (lysine) in said Motif I, and possesses both ssRNA ligase and ssDNA ligase activity at a temperature of at least 75° C.,
b) a first single stranded nucleic acid sequence with a 5′ end base that is adenylated; and
c) a second single stranded nucleic acid sequence with a 3′ end base.

17. The system of claim 16, further comprising a first container and a second container, and wherein said hyper-thermostable lysine-mutant ssDNA/RNA ligase is in said first container, and said first and/or second single-stranded nucleic acid sequence is in said second container.

18. The system of claim 16, wherein said 5′ end base of said first single stranded nucleic acid sequence is a DNA base, and wherein said 3′ end base of said second single stranded nucleic acid sequence is an RNA base or a DNA base.

19. The system of claim 16, wherein said 5′ end base of said first single stranded nucleic acid sequence is a DNA base or an RNA base, and wherein said 3′ end base of said second single stranded nucleic acid sequence is a DNA base.

20. The system of claim 16, wherein all of the bases in said first single stranded nucleic acid sequence are DNA bases.

21. The system of claim 16, wherein all of the bases in said second single stranded nucleic acid sequence are DNA bases.

22. The system of claim 16, wherein said amino acid substitution for said K (lysine) is an amino acid selected from the group consisting of: alanine (A), serine (S), cysteine (C), valine (V), threonine (T), and Glycine (G).

23. The system of claim 22, wherein said precursor hyper-thermostable ssRNA ligase is a wild-type hyper-thermostable ssRNA ligase.

24. The system of claim 23, wherein said wild-type hyper-thermostable ssRNA ligase is from a species selected from the group consisting of: Thermococcus kodakarensis, Pyrococcus, yayanosii, Pyrococcus horikoshii, Pyrococcus abyssi, Pyrococcus furiosus, Hyperthermus butylicus, Aeropyrum pernix, Staphylothermus marinus, Pyrolobus fumarii, and Aquifex aeolicus.

25. The system of claim 16, wherein said hyper-thermostable lysine-mutant ssDNA/RNA ligase is encoded by one of the amino acid sequences shown in SEQ ID NO:1-11.

26. The system of claim 16, wherein said hyper-thermostable lysine-mutant ssDNA/RNA ligase is encoded by an amino acid sequence that has at least 95% sequence identity to one of the amino acid sequences shown in SEQ ID NO:1-11.

27. The system of claim 16, wherein said first single stranded nucleic acid sequence is a sequencing adapter.

28. The system of claim 16, wherein said second single stranded nucleic acid sequence comprises a sequencing library fragment.

29. A composition comprising: a hyper-thermostable lysine-mutant ssDNA/RNA ligase which is a mutated version of a precursor hyper-thermostable ssRNA ligase,
wherein said precursor hyper-thermostable ssRNA ligase has a Motif I EKx(D/N/H)G (SEQ ID NO:32) and possess ssRNA ligase activity, but not ssDNA ligase activity, at a temperature of at least 75° C., and
wherein said hyper-thermostable lysine-mutant ssDNA/RNA ligase has an amino
acid substitution at said K (lysine) in said Motif I, and possesses both ssRNA ligase and ssDNA ligase activity at a temperature of at least 75° C.

30. The composition of claim 29, further comprising at least one of the following:
a) a first single stranded nucleic acid sequence with a 5′ end base that is adenylated, and
b) a second single stranded nucleic acid sequence with a 3′ end base.

31. The composition of claim 29, wherein said amino acid substitution for said K (lysine) is an amino acid selected from the group consisting of: alanine (A), serine (S), cysteine (C), valine (V), threonine (T), and Glycine (G).

32. The composition of claim 29, wherein said precursor hyper-thermostable ssRNA ligase is a wild-type hyper-thermostable ssRNA ligase.

33. The composition of claim 32, wherein said wild-type hyper-thermostable ssRNA ligase is from a species selected from the group consisting of: Thermococcus kodakarensis, Pyrococcus, yayanosii, Pyrococcus horikoshii, Pyrococcus abyssi, Pyrococcus furiosus, Hyperthermus butylicus, Aeropyrum pernix, Staphylothermus marinus, Pyrolobus fumarii, and Aquifex aeolicus.

34. The composition of claim 29, wherein said hyper-thermostable lysine-mutant ssDNA/RNA ligase is encoded by one of the amino acid sequences shown in SEQ ID NO:1-11.

35. The composition of claim 29, wherein said hyper-thermostable lysine-mutant ssDNA/RNA ligase is encoded by an amino acid sequence that has at least 95% sequence identity to one of the amino acid sequences shown in SEQ ID NO:1-11.

36. A method comprising:
a) combining in a reaction mixture:
i) a single-stranded RNA ligase from Pyrococcus furiousus (PfuRnl2), and
ii) a single-stranded nucleic acid sequence with an un-adenylated 5′ end,
iii) ATP molecules; and
b) incubating said reaction mixture under condition such that said PfuRnl2 adenylates said un-adenylated 5′ end of said single-stranded nucleic acid sequence, thereby generating a first single stranded nucleic acid sequence with a 5′ end base that is adenylated.

37. The method of claim 36, wherein said incubating is at a temperature of at least 75° C.

38. The method of claim 36, wherein said combining in said reaction mixture further includes: iv) a second single stranded nucleic acid sequence with a 3′ end base, v) a hyper-thermostable lysine-mutant ssDNA/RNA ligase which is a mutated version of a precursor hyper-thermostable ssRNA ligase, wherein said precursor hyper-thermostable ssRNA ligase has a Motif I EKx(D/N/H)G (SEQ ID NO:32) and possess ssRNA ligase activity, but not ssDNA ligase activity, at a temperature of at least 75° C., and wherein said hyper-thermostable lysine-mutant ssDNA/RNA ligase has an amino acid substitution at said K (lysine) in said Motif I, and possesses both ssRNA ligase and ssDNA ligase activity at a temperature of at least 75° C.

39. The method of claim 38, wherein said hyper-thermostable lysine-mutant ssDNA/RNA ligase ligates said 5′ adenylated end of said first single stranded nucleic acid sequence to said 3′ end of said second single stranded nucleic acid sequence to form a ligated nucleic acid sequence.

40. A composition, kit, or system comprising:
a) a single-stranded RNA ligase from Pyrococcus furiousus (PfuRnl2), and
b) a hyper-thermostable lysine-mutant ssDNA/RNA ligase which is a mutated version of a precursor hyper-thermostable ssRNA ligase, wherein said precursor hyper-thermostable ssRNA ligase has a Motif I EKx(D/N/H)G (SEQ ID NO:32) and possess ssRNA ligase activity, but not ssDNA ligase activity, at a temperature of at least 75° C., and wherein said hyper-thermostable lysine-mutant ssDNA/RNA ligase has an amino acid substitution at said K (lysine) in said Motif I, and possesses both ssRNA ligase and ssDNA ligase activity at a temperature of at least 75° C.

41. The composition of claim 40, further comprising: c) a single-stranded nucleic acid sequence with an un-adenylated 5′ end.


SEQUENCE LISTING (32 sequences)

SEQ ID NO: 1 - protein, 380 aa, Thermococcus kodakarensis
  misc_feature (92)..(92): Xaa can be any naturally occurring amino acid, except K
MVSSYFRNLLLKLGLPEERLEVLEGKGALAEDEFEGIRYVRFRDSARNFRRGTVVFETGEAVLGFPHIKRVVQLENGIRRVFKNKPFYVEEXVDGYNVRVVKVKDKILAITRGGFVCPFTTERIEDFVNFDFFKDYPNLVLVGEMAGPESPYLVEGPPYVKEDIEFFLFDIQEKGTGRSLPAEERYRLAEEYGIPQVERFGLYDSSKVGELKELIEWLSEEKREGIVMKSPDMRRIAKYVTPYANINDIKIGSHIFFDLPHGYFMGRIKRLAFYLAENHVRGEEFENYAKALGTALLRPFVESIHEVANGGEVDETFTVRVKNITTAHKMVTHFERLGVKIHIEDIEDLGNGYWRITFKRVYPDATREIRELWNGLAFVD

SEQ ID NO: 2 - protein, 379 aa, Pyrococcus yayanosii
  misc_feature (92)..(92): Xaa can be any naturally occurring amino acid, except K
MVSSHFKEILMRLGLPEDRIEVLEAKGGITEEEFDGIRYLRFKDSARGLRRGTVVFDEANVILGFPHIKRVVSLRAGVMRIFKRTPFYVEEXVDGYNVRVALVSDRVLAITRGGFVCPFTTERILDFVPEEFFKDYPHLVLVGEMAGPESPYLVEGPPYVEEDIRFFLFDIQEKGTGKSLPVQERLKLAEEYGIPHVKVFGLYTVDRIEDLYDLIERLSREGREGVVMKSPDMKRVVKYVTPFANVNDVKIGAKVFFELPPGYFMSRIMRLAFYVAERRIKGERFEELARNLGKALLEPFVESIWDVEQGDEIAEVFRIRVKRIETAYKMVTHFERLGLNIKIEDIEEVGGMWRITFKRAYDEATREIRELIGGRAFVD

SEQ ID NO: 3 - protein, 379 aa, Pyrococcus horikoshii
  misc_feature (92)..(92): Xaa can be any naturally occurring amino acid, except K
  misc_feature (107)..(107): Xaa can be any naturally occurring amino acid, except K
MVSSKFKDILYRLGIPEGKVEDLEARGGLVEDKFDDIKYLRIRNSVGKLRRGTVVLNDKFIILGFPHIKRIVNLKNGIKRTFKRGEFYVEEXVDGYNVRVVKFRGKXLGITRGGFICPFTTERISDFIPEEFFKDHPNLILVGEMAGPESPYLVEGPPYVKEDIQFFLFDIQELGTGRSLPVEERLKIAEEYGISHVEVFGKFTYKDLEEIYEIVERLSREGREGIVMKSPDMRKMVKYVTPYANINDIKIGARVFYELPPGYFTSRISRLAFYIAEKRLRGENFEELAKELGKALLQPLVESIHDVEQEDEIAEVFKVRVKKIETAYKMVTHFEKLGLRIEIVDIEEMKGGWRITFKRLYPDATEEIRELIGGKSFVD

SEQ ID NO: 4 - protein, 382 aa, Pyrococcus abyssi
  misc_feature (95)..(95): Xaa can be any naturally occurring amino acid, except K
MKEVVSSVYKEILVKLGLTEDRIETLEMKGGIIEDEFDGIRYVRFKDSAGKLRRGTVVIDEEYVIPGFPHIKRIINLRSGIRRIFKRGEFYVEEXVDGYNVRVVMYKGKMLGITRGGFICPFTTERIPDFVPQEFFKDNPNLILVGEMAGPESPYLVEGPPYVKEDIQFFLFDVQEIKTGRSLPVEERLKIAEEYGINHVEVFGKYTKDDVDELYQLIERLSKEGREGIIMKSPDMKKIVKYVTPYANINDIKIGARVFYELPPGYFTSRISRLAFYLAEKRIKGEEFERVAKELGSALLQPFVESIFDVEQEEDIHELFKVRVKRIETAYKMVTHFEKLGLKIEIVDIEEIKDGWRITFKRLYPDATNEIRELIGGKAFVD

SEQ ID NO: 5 - protein, 382 aa, Pyrococcus furiosus
  misc_feature (95)..(95): Xaa can be any naturally occurring amino acid, except K
MENMVSSKFKELLYTLGIPEDKVEILEARGGIMEDEFEGIRYLRFKNSVGKLRRGTVLFEDGTTVFGFPHIKRIVNLSAGVRKIFKSSEFYVEEXVDGYNVRVVKFKDRILGITRGGFICPYTTERIAEFVPEEFFKDHKDLVLVGEMAGPESPYLVEGPPYVKEDIQFFLFDIQDIKTGSSLPVEERLKLAEEYGINHVEVFGRYSYKDIDDLYELIERLSREGREGIVMKSPDMKKIVKYVTPYANINDIKIGARVFYELPGGYFTSRISRLAFYIAEKKIRGEELHNLALQLGKALLQPLVEAIHDVTQGDVIAERFRVRVRKIETAYKMVTHFEKLGLEIEIEDIEEIEGGWRVTFKRVYPEATREIRDLIGGKAFVD

SEQ ID NO: 6 - protein, 390 aa, Hyperthermus butylicus
  misc_feature (100)..(100): Xaa can be any naturally occurring amino acid, except K
MTWIKNPEPWMVNLVAEKLGLDVERVETLARHGTIRFRGYRDVVYALLRREIAGHPEGTVVLLERNGARLVPGYPPIQRMVLPTIALPRHFIDKVVVEEXMNGYNVRLVMFHRKLLAVTRGGFICPYTTARLERLIGGRVRELFREIDPETYTIAGEVVGLENPYTRYFYPEAPRFDYFVFDLFHELKPLPPLERNELLEKYGLKHVRLLGVIDKNDVEMFKQIVAELDREGREGVVAKDPEYRVPPLKYTTSAVNIGDVRYGMRFFMEEGRSFLFSRLLRELFRAYEEGFGDAQLEKLALEFGRAATEPALESIRKVAMGDMLYEEFELVFADEVELEEFTSYMAELGVDIVVVSTSREDEGLRARMRKIKDTWIQLRKVLDTGLSPVD

SEQ ID NO: 7 - protein, 390 aa, Artificial sequence
  Synthetic peptide
MTWIKNPEPWMVNLVAEKLGLDVERVETLARHGTIRFRGYRDVVYALLRREIAGHPEGTVVLLERNGARLVPGYPPIQRMVLPTIALPRHFIDKVVVEEAMNGYNVRLVMFHRKLLAVTRGGFICPYTTARLERLIGGRVRELFREIDPETYTIAGEVVGLENPYTRYFYPEAPRFDYFVFDLFHELKPLPPLERNELLEKYGLKHVRLLGVIDKNDVEMFKQIVAELDREGREGVVAKDPEYRVPPLKYTTSAVNIGDVRYGMRFFMEEGRSFLFSRLLRELFRAYEEGFGDAQLEKLALEFGRAATEPALESIRKVAMGDMLYEEFELVFADEVELEEFTSYMAELGVDIVVVSTSREDEGLRARMRKIKDTWIQLRKVLDTGLSPVD

SEQ ID NO: 8 - protein, 385 aa, Aeropyrum pernix
  misc_feature (99)..(99): Xaa can be any naturally occurring amino acid, except K
MASAAEVLASALRAVGVDPGSVDLEALSTRRSVRVSRFEDVVYVGFRRQFRGVPEGTLVAFRRGEQIVVWGYPSIKRMLLPRVAVPRWFPGPTVLVEEXMNGYNVRVFTLGGMVYAATRGGLICPYTTRRLRRLYGGALQKILEDLGAEGSFIAGEVVGLENPYTRYYYEEAPGFGYFIFDIFKGGRQLPPRVKFSLAPEYGLKTVNLLAEIPATASGVERLYTIVEDLEKRGREGVIVKDPEGRVEPLKYTTSRTNIGDIRLGMRYPFEEGRSFLFPRILREIFREWETGRRRYGELGEAILAPAIEAVEAVSRGGRLVEEFELVFANEVEAEEVIAYFASLGVHLEIAGVARGVDGVRVAFRKPRKSEGEIARILETGISPLD

SEQ ID NO: 9 - protein, 381 aa, Staphylothermus marinus
  misc_feature (92)..(92): Xaa can be any naturally occurring amino acid, except K
MDENELVNKLSDALGIEYEKLSKHIGRSIRLMKYGELNYVVFRRDLLGYREGTTILLGEEPLIVHGYPSIQRLAFIEGVSKHMIDNVVVEEXMNGYNVRVVYYMNNIYAITRGGYICPYTTARIRKLYSKNIKLAYQEYPDTILVGEVVGTENPYVIYDYPEARGFDYFIFDTMKKDKLQPLRIRDEIAEKYSLKTVRILDIINKRDIDRLKTIINRLEKERREGVVLKDPYQRVPPLKYTTIYINIRDIWEGMRYPFDEGRGYLFSRIVRLIAQGYEYDWNNTELDRIALKLGRAILEPAINSLKKRANGEIIASKYTLVFPSEDDLSKYIEYAESIGMDFIFRVVEKREDGCIVVELFKMKETHNIYTKMLKTGYSPLD

SEQ ID NO: 10 - protein, 390 aa, Pyrolobus fumarii
  misc_feature (100)..(100): Xaa can be any naturally occurring amino acid, except K
MIRIPLERWMIEKLAEALNVNIEEAERLARRRNVVRLMKWRNVTYFSLRKDVYGLREGTLIAVWPDGYRVVPGYPSIQRVLLPSVALPKHFIDKIVVEEXLNGYNVRVVKLRDEIVAVTRGGLICPYTTQRIRKLYGDKLTSLFREEGEELVVAGEVIGLENPYVRFYYPEAGGFAYFIFDIVHGEKFLPPHERKEIVEKHGLLHVPVLGEIDKNDIKAFRKIIEDLERRGREGVVLKDPEYRVPPLKYTTSFINIHDIEIGMRFPFDEGRNYLFSRILREIFKAVEEGWDDRRLLLAEQNLGKAILEPAIEAVKEVKNGKMLYEEFMLPFDTRDDFEEFLDYMASLGVDIIVAGVEQRSDGSIVARIRKVKDTWREVQKILETGLSPID

SEQ ID NO: 11 - protein, 367 aa, Aquifex aeolicus
  misc_feature (77)..(77): Xaa can be any naturally occurring amino acid, except K
MISPELVKEALKKKKVRSEEAFGLEYLRFNDDYKDIPRGTAIFKDFIIWGYPHIGRIFLLETGLREQFEAPFWVEEXVDGYNTRIFKYGDNYYALSRGGFICPFTTDRLPDLIDLRILDENPDLVICAEVAGPENPYIEESPPYVKEDVQLFVFDFMKKNEQGFLSQEEKMELIEKYNLPHVEILGRFTASEEGIKKIKEILKRFNEEGREGVVFKEDSERNKRAKYITSYANLMDIKTNAKNMLQLPPEYYTNRILRLVLFMYEEGLKTTEHLYEELGRAFIDGLFQAIEQFEKEHKVYKTFTCKFRKKENAIALLELLSKTSKHIQVKERRLEKEGDYWRLEFDKVFLNMTGLLGHLLSGGIVYD

SEQ ID NO: 12 - protein, 5 aa, Artificial sequence
  Synthetic peptide
  MISC_FEATURE (3)..(3): Xaa = any amino acid
  MISC_FEATURE (4)..(4): Xaa = D, N, or H
EGXXG

SEQ ID NO: 13 - protein, 5 aa, Artificial sequence
  Synthetic peptide
  MISC_FEATURE (3)..(3): Xaa = any amino acid
  MISC_FEATURE (4)..(4): Xaa = D, N, or H
EPXXG

SEQ ID NO: 14 - protein, 5 aa, Artificial sequence
  Synthetic peptide
  MISC_FEATURE (3)..(3): Xaa= any amino acid
  MISC_FEATURE (4)..(4): Xaa = D, N, or H
EAXXG

SEQ ID NO: 15 - protein, 5 aa, Artificial sequence
  Synthetic peptide
  MISC_FEATURE (3)..(3): Xaa = any amino acid
  MISC_FEATURE (4)..(4): Xaa = D, N, or H
EVXXG

SEQ ID NO: 16 - protein, 5 aa, Artificial sequence
  Synthetic peptide
  MISC_FEATURE (3)..(3): Xaa = any amino acid
  MISC_FEATURE (4)..(4): Xaa = D, N, or H
ELXXG

SEQ ID NO: 17 - protein, 5 aa, Artificial sequence
  Synthetic peptide
  MISC_FEATURE (3)..(3): Xaa = any amino acid
  MISC_FEATURE (4)..(4): Xaa = D, N, or H
EIXXG

SEQ ID NO: 18 - protein, 5 aa, Artificial sequence
  Synthetic peptide
  MISC_FEATURE (3)..(3): Xaa = any amino acid
  MISC_FEATURE (4)..(4): Xaa = D, N, or H
EMXXG

SEQ ID NO: 19 - protein, 5 aa, Artificial sequence
  Synthetic peptide
  MISC_FEATURE (3)..(3): Xaa = any amino acid
  MISC_FEATURE (4)..(4): Xaa = D, N, or H
ECXXG

SEQ ID NO: 20 - protein, 5 aa, Artificial sequence
  Synthetic peptide
  MISC_FEATURE (3)..(3): Xaa = any amino acid
  MISC_FEATURE (4)..(4): Xaa = D, N, or H
EFXXG

SEQ ID NO: 21 - protein, 5 aa, Artificial sequence
  Synthetic peptide
  MISC_FEATURE (3)..(3): Xaa = any amino acid
  MISC_FEATURE (4)..(4): Xaa = D, N, or H
EYXXG

SEQ ID NO: 22 - protein, 5 aa, Artificial sequence
  Synthetic peptide
  MISC_FEATURE (3)..(3): Xaa = any amino acid
  MISC_FEATURE (4)..(4): Xaa = D, N, or H
EWXXG

SEQ ID NO: 23 - protein, 5 aa, Artificial sequence
  Synthetic peptide
  MISC_FEATURE (3)..(3): Xaa = any amino acid
  MISC_FEATURE (4)..(4): Xaa = D, N, or H
EHXXG

SEQ ID NO: 24 - protein, 5 aa, Artificial sequence
  Synthetic peptide
  MISC_FEATURE (3)..(3): Xaa = any amino acid
  MISC_FEATURE (4)..(4): Xaa = D, N, or H
ERXXG

SEQ ID NO: 25 - protein, 5 aa, Artificial sequence
  Synthetic peptide
  MISC_FEATURE (3)..(3): Xaa = any amino acid
  MISC_FEATURE (4)..(4): Xaa = D, N, or H
EQXXG

SEQ ID NO: 26 - protein, 5 aa, Artificial sequence
  Synthetic peptide
  MISC_FEATURE (3)..(3): Xaa= any amino acid
  MISC_FEATURE (4)..(4): Xaa = D, N, or H
ENXXG

SEQ ID NO: 27 - protein, 5 aa, Artificial sequence
  Synthetic peptide
  MISC_FEATURE (3)..(3): Xaa = any amino acid
  MISC_FEATURE (4)..(4): Xaa = D, N, or H
EEXXG

SEQ ID NO: 28 - protein, 5 aa, Artificial sequence
  Synthetic peptide
  MISC_FEATURE (3)..(3): Xaa = any amino acid
  MISC_FEATURE (4)..(4): Xaa = D, N, or H
EDXXG

SEQ ID NO: 29 - protein, 5 aa, Artificial sequence
  Synthetic peptide
  MISC_FEATURE (3)..(3): Xaa = any amino acid
  MISC_FEATURE (4)..(4): Xaa = D, N, or H
ESXXG

SEQ ID NO: 30 - protein, 5 aa, Artificial sequence
  Synthetic peptide
  MISC_FEATURE (3)..(3): Xaa = any amino acid
  MISC_FEATURE (4)..(4): Xaa = D, N, or H
ETXXG

SEQ ID NO: 31 - protein, 4 aa, Artificial sequence
  Synthetic peptide
  MISC_FEATURE (2)..(2): Xaa = any amino acid
  MISC_FEATURE (3)..(3): Xaa = D, N, or H
EXXG

SEQ ID NO: 32 - protein, 5 aa, Artificial sequence
  Synthetic peptide
  MISC_FEATURE (3)..(3): Xaa = any amino acid
  MISC_FEATURE (4)..(4): Xaa = D, N, or H
EKXXG